(19) United States
(12) Patent Application Publication
(10) Pub. No.: US 2005/0192547 A1
(43) Pub. Date: Sep. 1, 2005

(54) Combinations of antiseptic and antibiotic agents containing medical devices

(76) Inventors: Shanta M. Modak, River Edge, NJ (US); Suhas Tambe, Mumbai (IN); Lester A. Sampath, Nyack, NY (US)

Correspondence Address: BAKER & BOTTS, 30 ROCKEFELLER PLAZA, NEW YORK, NY, 10112, US

(21) Appl. No.: 10/633,204

(22) Filed: Jul. 30, 2003

Related U.S. Application Data
(63) Continuation of application No. PCT/US02/03087, filed on Jan. 31, 2002.

(51) Int. Cl. A61M 25/00

(52) U.S. Cl. 604/265

(57) ABSTRACT

The present invention relates to compositions comprising a combination of one or more antiseptic and an antibiotic. It is based, at least in part, on the discovery that such combinations tend to deter the formation of antibiotic-resistant organisms. In preferred, nonlimiting embodiments of the invention, the antibiotic is minocycline and the antiseptic is a chlorhexidine compound, triclosan, or benzalkonium chloride, and in particular embodiments, a silver salt or a bismuth salt is added. Examples of specific, nonlimiting embodiments of the invention include combinations of (i) minocycline, triclosan, and a bismuth salt; (ii) minocycline, a chlorhexidine compound, and a bismuth salt; and (iii) minocycline, benzalkonium chloride, and a bismuth salt. The present invention further provides for articles, such as, but not limited to, medical articles, which have been treated with or which otherwise comprise a combination of antiseptic and antibiotic.

The present invention relates to combinations of antiseptic and antibiotic agents which exert an antimicrobial effect while deterring, relative to other antimicrobial agents, the development of antibiotic-resistant microorganisms.

### BACKGROUND OF THE INVENTION

An antiseptic is a substance that kills or prevents the growth of microorganisms, and which is typically applied to living tissue, distinguishing the class from disinfectants, which are usually applied to inanimate objects (Goodman and Gilman's “The Pharmacological Basis of Therapeutics”, Seventh Edition, Gilman et at., editors, 1985, Macmillan Publishing Co., (hereafter, Goodman and Gilman”) pp. 959-960). Common examples of antiseptics are ethyl alcohol and tincture of iodine. Alcohol is usually used to clean a subject's skin prior to insertion of a hypodermic needle; tincture of iodine is frequently applied as a first step in wound care, both uses intended to decrease the number of microbes on the skin to prevent infection.

While antiseptics once played a more substantial role in wound management, they are now secondary in importance to antibiotics, chemical substances produced by various species of microorganisms (or synthetic or semisynthetic analogs thereof) that kill or suppress the growth of other microorganisms (Goodman and Gilman, p.

1067). Antibiotics may be administered systemically or locally applied. Since the production of penicillin in 1941, antibiotics have been widely used, with the result that microorganism strains have developed which are resistant to one or more antibiotic. The generation of resistant organisms has created an ever-increasing need for the identification or synthesis of new antibiotics (Goodman and Gilman, p. 1066).

One particularly useful class of antibiotics is tetracyclines, which exert their antibacterial action by binding to microbial ribosomes and preventing protein synthesis (Goodman and Gilman, p. 1171). Tetracyclines are primarily bacteriostatic when tested in vitro, and only multiplying microorganisms are affected. They possess antimicrobial activity against a wide variety of microorganisms, including gram-positive and gram negative bacteria, and against some microorganisms, such as Rickettsiae, Mycoplasma, Chlamydia, some atypical Mycobacteria, and ameobae, that are resistant to other classes of antibiotics (Id.). Minocycline, doxycycline, tetracycline and oxytetracycline are tetracycline-class drugs listed in order of decreasing antimicrobial activity (Id.).

The first documented medical use of bismuth occurred in 1773, when it was used in salves. Since then it has been used to combat diarrhea, gastroenteritis, stomach cramps, vomiting and ulcers. It has been used for the treatment of surgical wounds (Bierer, 1990, Rev. Infect. Dis. 12 (Suppl. 1): S3-S8). The antimicrobial effect of bismuth is well known. The reducing effect of bismuth compounds on fermentation by colonic bacteria has been demonstrated both in vitro and in vivo (Leon-Barua et al., 1990, Rev. Infect. Dis. 12 (Suppl. 1): S24-S29).

Bismuth salts have also been shown to have a significant effect on the inhibitory activity of antibiotics. Bismuth salicylate (“BSS”) and bismuth nitrate have been reported to potentiate aminoglycoside activity against gram negative bacteria (Domenico et al., 1991, J. Antimicrob. Chemother. 28:801-810). BSS, bismuth nitrate and bismuth dimercaprol (Bis-BAL), have also been reported to inhibit capsular polysaccharide production by the bacterium Klebsiella pneumoniae (Domenico et al., 1991, J. Antimicrobial Chemother. 28:801-810; Domenico et al., 1996, Ann. N.Y. Acad. Sci. 797:269-270). BisBAL has also been shown to have good activity against a wide spectrum of bacteria among the genera Yersinia, Shigella, Salmonella, Pseudomonas, Proteus, Enterobacter, Escherichia, Staphylococci, Helicobacter, and Clostridia (Domenico, 1997, Antimicrob. Ag. Chemother. 41:1697-1703) and to inhibit polysaccharide production in biofilms (Huang and Stewart, 1999, J. Antimicrob. Chemother. 44:601-605). The use of bismuth salts in combination with thiol compounds for the preparation of a composition with anti-infective properties has been described by Domenico, 1999, U.S. Pat. No. 5,928,671. The potentiation of antibiotics by a bismuth salt of pyrrolidone carboxylic acid, resulting in higher tissue levels of the antibiotic was described by Bocher et al., 1977, U.S. Pat. No. 4,064,238. The use of bismuth salts, in combination with an antibiotic and metronidazole for the eradication of Helicobacter pylori, a causative agent of duodenal ulcer, has also been described (Borody, 1993, U.S. Pat. No. 5,196,205).

International Application No. PCT/US00/08692, entitled “TRICLOSAN AND SILVER COMPOUND CONTAINING MEDICAL DEVICES” by The Trustees of Columbia University in the City of New York teaches the addition of various antibiotics, including those set forth herein, to combinations of triclosan and silver salts. The use of bismuth salts, as set forth herein, was not disclosed.

### SUMMARY OF THE INVENTION

The present invention relates to compositions comprising a combination of one or more antiseptic and an antibiotic. It is based, at least in part, on the discovery that such combinations tend to deter the formation of antibiotic-resistant organisms. In preferred, nonlimiting embodiments of the invention, the antibiotic is minocycline and the antiseptic is a chlorhexidine compound, triclosan, or benzalkonium chloride, and in particular embodiments, a silver salt or a bismuth salt is added. Examples of specific, nonlimiting embodiments of the invention include combinations of (i) minocycline, triclosan, and a bismuth salt; (ii) minocycline, a chlorhexidine compound, and a bismuth salt; and (iii) minocycline, benzalkonium chloride, and a bismuth salt. The present invention further provides for articles, such as, but not limited to, medical articles, which have been treated with or which otherwise comprise a combination of antiseptic and antibiotic.

Antibiotics, unlike antiseptics, may be used in relatively high concentrations because they tend to be less toxic to host tissue. The use of higher concentrations may result in longer term efficacy. In contrast, antiseptics typically should be used in lower concentrations, because they are frequently toxic to host tissue. Even at lower concentrations, however, antiseptics may provide cidal action against a wide range of microorganisms. Thus, combinations of antibiotics and antiseptics according to the invention may provide for prolonged antimicrobial effectiveness against a variety of microbes.

### DETAILED DESCRIPTION OF THE INVENTION

The present invention relates to compositions comprising combinations of (i) an antibiotic selected from the group consisting of minocycline, rifampin, and norfloxacin; and (ii) an antiseptic selected from the group consisting of biguanide compounds, triclosan, and benzalkonium chloride. In preferred embodiments, such compositions further comprise a salt of bismuth, cerium, or zinc or a silver-containing compound. Such compositions may be incorporated into or onto medical devices to impart antimicrobial activity to the devices. The present invention also provides for methods of using such compositions in the preparation of medical devices.

Biguanide compounds which may be used according to the invention include poly (hexamethylene biguanide) hydrochloride and chlorhexidine compounds. Chlorhexidine is the term denoting the chemical compound 1,6 bis(N⁵-p-chlorophenyl-N¹-biguanido)hexane). Chlorhexidine compounds include chlorhexidine free base (“CHX”) as well as chlorhexidine salts, such as chlorhexidine diphos-phanilate, chlorhexidine digluconate (“CHG”), chlorhexidine diacetate (“CHA”), chlorhexidine dihydrochloride, chlorhexidine dichloride, chlorhexidine dihydroiodide, chlorhexidine diperchlorate, chlorhexidine dinitrate, chlorhexidine sulfate, chlorhexidine sulfite, chlorhexidine thiosulfate, chlorhexidine di-acid phosphate, chlorhexidine difluoro-phosphate, chlorhexidine diformate, chlorhexidine dipro-pionate, chlorhexidine di-iodobutyrate, chlorhexidine di-n-valerate, chlorhexidine dicaproate, chlorhexidine malonate, chlorhexidine succinate, chlorhexidine malate, chlorhexidine tartrate, chlorhexidine dimonoglycolate, chlorhexidine mono-diglycolate, chlorhexidine dilactate, chlorhexidine di-y-hydroxyisobutyrate, chlorhexidine diglucoheptonate, chlor-hexidine di-isothionate, chlor-hexidine dibenzoate, chlor-hexidine dicinnamate, chlor-hexidine dimandelate, chlor-hexidine di-isophthalate, chlorhexidine di-2-hydroxy-napthoate, and chlorhexidine embonate.

Bismuth salts which may be used according to the invention include bismuth nitrate, bismuth citrate, bismuth salicylate, bismuth borate, bismuth mandelate, bismuth palmitate, bismuth benzoate, and bismuth sulfadiazine.

Cerium salts which may be used according to the invention include cerium nitrate and other cerium salts having a water solubility similar to cerium nitrate.

The term silver-containing compound, as used herein, refers to a compound comprising silver, either in the form of a silver atom or a silver ion unlinked or linked to another molecule via a covalent or noncovalent (e.g., ionic) linkage, including but not limited to covalent compounds such as silver sulfadiazine (“AgSD”) and silver salts such as silver oxide (“ Ag₂O”), silver carbonate (“Ag₂CO₃”), silver deoxycholate, silver salicylate, silver iodide, silver nitrate (“AgNO₃”), silver paraminobenzoate, silver paraminosalicylate, silver acetylsalicylate, silver ethylenediaminetetraacetic acid (“Ag EDTA”), silver picrate, silver protein, silver citrate, silver lactate and silver laurate.

Zinc salts which may be used according to the invention include zinc acetate and other zinc salts having a water solubility similar to zinc acetate.

The present invention provides for the incorporation of combinations of the foregoing elements into or onto medical devices, and for the medical devices which incorporate said combinations. The terms “medical article” and “medical device” are used interchangeably herein. Medical articles that may be treated according to the invention are either fabricated from or coated or treated with biomedical polymer (and hence may be referred to as “polymer-containing medical articles”) and include, but are not limited to, catheters including urinary catheters and vascular catheters (e.g., peripheral and central vascular catheters), wound drainage tubes, arterial grafts, soft tissue patches (such as polytetrafluoroethylene (“PTFE”) soft tissue patches), gloves, shunts, stents, tracheal catheters, wound dressings, sutures, guide wires and prosthetic devices (e.g., heart valves and LVADs). Vascular catheters which may be prepared according to the present invention include, but are not limited to, single and multiple lumen central venous catheters, peripherally inserted central venous catheters, emergency infusion catheters, percutaneous sheath introducer systems and thermodilution catheters, including the hubs and ports of such vascular catheters.

In particular embodiments, the combinations of the invention may be incorporated into or onto a medical device by exposing the device to a treatment solution comprising the combination in an appropriate solvent system. Said treatment solutions fall within the scope of compositions covered by the present invention. Where the treatment solution contains minocycline, the concentration of minocycline is between 1 and 8 percent weight/volume (w/v), and preferably between 3 and 5 percent (w/v); where the treatment solution contains rifampin, the concentration of rifampin is between 1 and 8 percent (w/v), and preferably between 3 and 5 percent (w/v); where the treatment solution contains norfloxacin, the concentration of norfloxacin is between 1 and 8 percent (w/v) and preferably between 3 and 5 percent (w/v); where the solution contains a chlorhexidine compound, the concentration of chlorhexidine compound is between 1 and 8 percent (w/v) and preferably between 3 and 5 percent (w/v); where the treatment solution contains triclosan, the concentration is between 1 and 8 percent (w/v) and preferably between 3 and 5 percent (w/v); where the treatment solution contains benzalkonium chloride, the concentration of benzalkonium chloride (“BZK”) is between 0.25 and 1 percent (w/v) and preferably is 0.5 percent (w/v); where the treatment solution contains a bismuth salt, the concentration of bismuth salt is between 0.5 and 2 percent (w/v) and preferably is 2 percent (w/v); where the treatment solution contains a cerium salt, the concentration of cerium salt is preferably between 1 and 5 percent (w/v); where the treatment solution contains a zinc salt, the concentration of zinc salt is between 1 and 5 percent (w/v) and preferably is 2 percent (w/v); and where the treatment solution contains a silver-containing compound, the concentration of silver-containing compound is between 0.5 and 2 percent (w/v) and preferably is 1 percent. The above ranges, e.g. “between X percent and Y percent”, include the boundary values X and Y and are intended herein to encompass variations of 20 percent of the value of X and Y, in other words, the ranges should be interpreted to mean “between X±0.20X and Y±0.20Y”.

An appropriate solvent system is a solvent system which will either solubilize or, less desirably, produce a suspension of anti-infective agents, which will preferably result in slight swelling of the medical device (to facilitate incorporation of anti-infective agents), but which will preferably not substantially alter the surface of the medical device (e.g., render the surface rough) so as not to impair the clinical usefulness of the device. Specific non-limiting examples of solvent systems include 70 percent (volume/volume; “v/v”) tetrahydrofuran (“THF”) and 30 percent (v/v) methanol (“MeOH”), and more preferably, for dissolving an antibiotic, 50 percent (v/v) tetrahydrofuran (“THF”) and 50 percent (v/v) methanol. Such treatment solutions may further comprise a biomedical polymer. As specific, non-limiting examples, the treatment solution may comprise 3 percent (w/v) 93A polyurethane and 1 percent (w/v) 60D polyurethane or 1 percent (w/v) 93A polyurethane and 3 percent (w/v) 60D polyurethane.

For example, a polyurethane catheter may be exposed to a treatment solution of the invention (by dipping and/or drawing treatment solution through the catheter lumen) for between 2 and 200 seconds, and preferably between 2 and 100 seconds, and then dried at room temperature. A polyurethane catheter treated in this manner using 1:1 THF/MeOH as a solvent system would contain the following amounts of anti-infective substances (where the term “anti-infective” refers to antibiotics and antiseptics): where the catheter contains minocycline, the amount of minocycline is between 100 and 450 micrograms per centimeter; where the catheter contains rifampin, the amount of rifampin is between 100 and 450 micrograms per centimeter; where the catheter contains norfloxacin, the amount of norfloxacin is between 100 and 450 micrograms per centimeter; where the catheter contains a chlorhexidine compound, the amount of chlorhexidine compound is between 130 and 520 micrograms per centimeter; where the catheter contains triclosan, the amount is between 130 and 750 micrograms per centimeter; where the catheter contains benzalkonium chloride, the amount of benzalkonium chloride (“BZK”) is between 25 and 100 micrograms per centimeter; where the catheter contains a bismuth salt, the amount of bismuth salt is between 50 and 300 micrograms per centimeter; where the catheter contains a cerium salt, the amount of cerium salt is between 50 and 200 micrograms per centimeter; where the catheter contains a zinc salt, the amount of zinc salt is between 50 and 200 micrograms per centimeter; and where the catheter contains a silver-containing compound, the amount of silver-containing compound is between 25 and 300 micrograms per centimeter. Furthermore, the present invention provides for catheters and other medical devices comprising the abovementioned amounts of anti-infective agents in the inventive combinations whether they have been prepared using such a treatment solution or by other means, such as by extrusion, by “painting” a coating solution comprising the inventive combinations, by coating with a powder comprising the inventive combinations, etc.

Examples of combinations covered by the present invention include, but are not limited to, the following:
minocycline and bismuth;
minocycline and chlorhexidine free base;
minocycline and chlorhexidine diacetate;
minocycline and chlorhexidine digluconate;
minocycline and triclosan;
minocycline, chlorhexidine free base and bismuth nitrate;
minocycline, chlorhexidine diacetate, and bismuth nitrate;
minocycline, chlorhexidine digluconate, and bismuth nitrate;
minocycline, triclosan and bismuth nitrate;
minocycline, chlorhexidine free base, and benzalkonium chloride;
minocycline, chlorhexidine diacetate and benzalkonium chloride;
minocycline, chlorhexidine digluconate and benzalkonium chloride;
minocycline, triclosan and benzalkonium chloride;
minocycline, chlorhexidine free base, and benzalkonium chloride;
minocycline, chlorhexidine diacetate and benzalkonium chloride;
minocycline, chlorhexidine digluconate and benzalkonium chloride;
minocycline, triclosan and benzalkonium chloride;
minocycline, chlorhexidine free base, bismuth nitrate and benzalkonium chloride;
minocycline, chlorhexidine diacetate, bismuth nitrate and benzalkonium chloride;
minocycline, triclosan and silver carbonate;
minocycline, chlorhexidine digluconate, bismuth nitrate and benzalkonium chloride; and
minocycline, triclosan, bismuth nitrate and benzalkonium chloride.

### WORKING EXAMPLES

### Example

### Reduced Efficacy of Catheters Containing Antibiotics Against Antibiotic-resistant Bacteria

Recently catheters impregnated with a combination of two antibiotics, minocycline and rifampin, have been developed for clinical use. It was believed that development of resistance to these agents used in combination would be unlikely since each agent has a different mode of action, such that they might act synergistically. However, studies in which the catheters have been implanted into rats have shown that, over time, the catheters lose antimicrobial activity against bacterial strains exhibiting either low level resistance to this antibiotic combination or high level resistance to rifampin. In contrast, catheters impregnated with the antiseptics chlorhexidine and silver sulfadiazine were effective against these antibiotic resistant strains.

To describe these studies in greater detail, intravenous catheters treated with minocycline and rifampin (“MR”) or chlorhexidine and silver sulfadiazine (“AST”) were prepared as follows. MR polyurethane catheters were purchased from Cook Critical Care, Inc., and contain approximately 0.5 mg of minocycline and 0.5 mg of rifampin per centimeter. To prepare the AST catheters, polyurethane catheters were dipped in a treatment solution comprising 3 percent (w/v) chlorhexidine diacetate (“CHA”), 0.75 percent (w/v) silver sulfadiazine (“AgSD”), 3 percent (w/v) 93A polyurethane and 1 percent (w/v) 60D polyurethane, in a solvent system consisting of 70 percent (v/v) tetrahydrofuran and 30 percent (v/v) methanol at room temperature for 2-5 seconds.

Rats received subcutaneous implants of MR or AST catheter segments. At 7, 14, and 21 days post-implant, catheter segments were removed and then placed on agar plates seeded with either a parent Staphylococcus epidermidis strain (“S. epi-s”; ATCC Acc. No. 35983) or a rifampin resistant variant thereof (“R-r”) or a strain resistant to both minocycline and rifampin (“MR-r”). The zones of inhibition against these strains produced by the MR or AST catheter segments were measured, and the results are shown in Table I.

TABLE I
Effectiveness of Antimicrobial Catheters Against Sensitive
and Resistant Bacterial Strains
Zones of inhibition (mm)

| | INITIAL | | DAY 7 | | DAY 14 | | DAY 21 | |
|---|---|---|---|---|---|---|---|---|
| STRAIN | MR | AST | MR | AST | MR | AST | MR | AST |
| S.epi-s | 24 | 14 | 23 | 9 | 22.5 | 8 | 17.9 | 7.3 |
| MR-r | 22.3 | 14 | 21 | 9 | 14 | 8 | 10 | 7.5 |
| R-r | 21 | 13.3 | 9 | 9 | 7 | 8 | 6.8 | 7.8 |

To summarize, by day 7 the MR catheter's activity against the R-r strain was greatly reduced. At days 7 and 14 the zones of inhibition against both the MR-r and R-r strains were drastically reduced compared to those against the parent, non-resistant S. epidermidis strain. The activity of the AST catheters was unaffected regardless of the antibiotic resistance profile of the test organisms.

### Example

### Efficacy of Antibiotic/Antiseptic Combinations

Minimum inhibitory concentration (“MIC”): The MIC of each antimicrobial agent, singly and in combination, was determined using a liquid medium, namely Trypticase Soy Broth (“TSB”). The antimicrobial agents were serially diluted so that each tube contained 5 ml which was then inoculated with microbes at a concentration of either 10⁴ or 10⁶ colony forming unit (“cfu”) per milliliter. These levels of inoculum represent a low and mid-level range of numbers of organisms which may potentially colonize IV catheters. Tubes were incubated for 24 hours at 37° C. and checked for turbidity. The lowest concentration of drug with no visible turbidity was deemed the MIC.

Development of Resistance: S. epidermidis was evaluated in conjunction with various antimicrobial agents and combinations for the development of resistant organisms. Culture tubes containing 5 ml of TSB were inoculated to obtain approximately 1×10⁴ organisms per milliliter at drug concentrations ranging from 3 doubling dilutions above to 3 doubling dilutions below the MIC for each agent. The initial inoculum was prepared by growing the culture in TSB overnight. Tubes were incubated at 37° C. for 24 hours. The culture tube serially preceding the MIC-containing tube was diluted to 10⁵ cfu/ml and used for the next transfer. After 10 to 20 passages cultures below the MIC tube were subcultured on blood agar plates and stored for susceptibility tests. This experiment was repeated using a cell density of 10⁶ cfu/ml at every passage. The following antiseptics and antibiotics were tested singly and in combination for resistance development using the above test: chlorhexidine diacetate (“CHX”), triclosan (“T”), parachlorometaxylenol (“PCMX”), polyhexamethylenebiguanide (“PHMB”), minocycline (“M”), tobramycin (“Tb”), norfloxacin (“Nf”), minocycline and rifampin (“M+R”), chlorhexidine and norfloxacin (“CHX+Nf”), chlorhexidine and tobramycin (“CHX+Tb”), triclosan and minocycline (“T+M”), minocycline and parachloronietaxylenol (“PCMX+M”), polyhexamethylenebiguanide and minocycline (“PHMB+M”), and chlorhexidine and rifampin (“CHX+R”). The results of the studies are presented in Tables IIA, IIB and III.

TABLE IIA
MICs of Antibiotic and Antiseptic Before and After 10-20 Passages
Through Subinhibitory Concentrations Using an Inoculum Density
of 10⁴ cfu/ml

| | MIC (μg/ml) | | |
|---|---|---|---|
| | BEFORE | MIC (μg/ml) | INCREASE IN |
| | PASSAGE | AFTER PASSAGE | MIC (fold) |
| ANTIBIOTIC | | | |
| Minocycline (M) | 0.078 | 0.156 | 2 |
| Rifampin (R) | 0.0195 | 500 | 25,000 |
| Norfloxacin (Nf) | 0.05 | 2.0 | 40 |
| Tobramycin (Tb) | 0.025 | 0.5 | 20 |
| ANTISEPTIC | | | |
| Chlorhexidine | 0.5 | 1.0 | 2 |
| (CHX) | | | |
| Triclosan (T) | 0.35 | 2.5 | 7 |
| PHMB | 0.31 | 0.31 | 1.0 |
| PCMX | 125 | 125 | 1.0 |
| ANTIBIOTIC | | | |
| COMBINATION | | | |
| M + R (1:1)* | 0.019 | 0.31 | 16 |
| ANTISEPTIC | | | |
| ANTIBIOTIC | | | |
| COMBINATION | | | |
| CHX + M (1:1)* | .06 | 0.1 | 1.66 |
| CHX + R (3:1)* | 0.06 | 0.5 | 8.3 |
| CHX + NF (1:1)* | 0.015 | 0.015 | 1.0 |
| T + M (1:1)* | 0.0125 | 0.0125 | 1.0 |
| T + M (1.6:1)* | 0.0125 | 0.0125 | 1.0 |
| PHMB + M (1:1)* | 0.1 | 0.1 | 1.0 |
| PHMB + Tb (1:1)* | 0.1 | 0.2 | 2.0 |

*w/w

TABLE IIB
MIC (μg/ml) Initially and After 10 Transfers Through
Subinhibitory Concentrations Using An Inoculumn
Density of 10⁶ cfu/ml

| | MIC (cfu/ml) | MIC (cfu/ml) | |
|---|---|---|---|
| | BEFORE | AFTER | INCREASE IN MIC |
| GROUP | PASSAGE | PASSAGE | (fold) |
| Minocycline (M) | 0.125 | 0.5 | 4 |
| Triclosan (T) | 0.45 | 3.2 | 7 |
| M + T (1:1)* | 0.0125 | 0.0125 | 1 |
| M + R (1:1)* | 0.06 | 1.0 | 17 |

*w/w

Of all the antibiotics tested, minocycline appears to be less likely to develop resistant bacteria against lower challenges (10⁴ cfu/ml). However, at higher cell densities, the MIC of minocycline increased 4-fold. Combinations of antiseptics such as triclosan and antibiotics such as minocycline appear to prevent the development of minocycline resistant bacteria.

TABLE III
Synergistic Activity of Antibiotics and Antiseptics,
Inoculum of 10⁴ cfu/ml

| | | FRACTIONAL |
|---|---|---|
| | | INHIBITORY |
| COMPOUND | MIC (μg/ml) | CONCENTRATION |
| Triclosan | 0.35 | N.A. |
| Minocycline | 0.078 | N.A. |
| Triclosan + Minocycline | 0.015 + 0.015 | 0.235 |
| (1:1)* | | |
| Triclosan + Minocycline | 0.0077 + 0.0048 | 0.0835 |
| (1.6:1)* | | |
| Chlorhexidine | 0.5 | N.A. |
| Norfloxacin | 0.05 | N.A. |
| Chlorhexidine + Norfloxacin | 0.015 | 0.165 |
| (1:1)* | | |

*w/w.

The concentration of each agent in Table III in the combination is expressed as a fraction of the concentration that causes the same effect when the same agent is tested alone (i.e., its fractional inhibitory concentration). If the sum of the fractional inhibitory concentrations is less than one, then the combination is synergistic.

In conclusion, norfloxacin and minocycline were observed to exhibit synergy in combination with, respectively, chlorhexidine and triclosan, in addition to having lower increases in MIC after 20 passages through sub-inhibitory concentrations in vitro (see Table IIA).

### Example

### Antiseptic/Antibiotic-Treated Catheters

Method. Polyurethane catheter segments were treated with one of the following:
5 percent (w/v) tobramycin was suspended in 50 percent (v/v) methanol and 50 percent (v/v) tetrahydrofuran (THF) with 3 percent (w/v) 60D polyurethane and 1 percent (w/v) 93A polyurethane; 5 percent (w/v) norfloxacin was suspended in 50 percent (v/v) methanol and 50 percent (v/v) THF with 3 percent (w/v) 60D polyurethane and 1 percent (w/v) 93A polyurethane; or 5 percent (w/v) minocycline was dissolved in 50 percent (v/v) methanol and 50 percent (v/v) THF with 3 percent (w/v) 60D polyurethane and 1 percent (w/v) 93A polyurethane.

Treatment consisted of exposing the catheter segment to treatment solution for 2-5 seconds at room temperature, and then air-drying the segments. Then, 0.5 cm catheter segments were placed vertically on trypticase soy agar (“TSA”) plates seeded with 0.3 ml of 10⁸ cfu/ml of a test culture of Staphylococcus epidermidis. After incubation at 37° C. for 24 hours the zones of inhibition were measured, after which the catheter segments were transferred to fresh TSA plates seeded with the same culture. Transfers were done every 24 hours during the study.

Results. Results are presented in Table IV.

TABLE IV
Zones of Inhibition (mm)

| CATHETER GROUP | DAY 1 | DAY 4 | DAY 7 |
|---|---|---|---|
| Tobramycin | >25 | 7 | 0 |
| Norfloxacin | >25 | 10 | 8 |
| Minocycline | >25 | 26 | 15 |

Zone sizes smaller than 15 mm indicate that the treatment with antibiotic may not be effective in preventing S. epidermidis adherence to catheters (Sheretz et al., 1993, J. Infect. Dis. 167:98-106).

Discussion. Based on the data presented in Tables IIA, IIB and III, norfloxacin, tobramycin, and minocycline, in combination with chlorhexidine or triclosan, appear to have the lowest increase in MIC after 20 transfers through sub-inhibitory concentrations of drugs. However, in order for antimicrobial activity of a medical device to be effective for an extended period of time, an incorporated antibiotic and/or antiseptic should be released slowly and steadily. The data presented in Table IV suggests that for catheters, the choice of antibiotics for incorporation should be limited to minocycline and rifampin, because the antibiotics norfloxacin and tobramycin diffuse out of a catheter in a day or two, whereas minocycline and rifampin are released at a slower rate over a longer period of time. Further, norfloxacin and tobramycin have disadvantageous solubility characteristics, having limited solubility in the tetrahydrofuran/methanol solvent systems used to impregnate polyurethane catheters. This low solubility results in an upper limit of norfloxacin or tobramycin incorporated of about 0.5 percent (w/v), a level associated with short-lived, low antimicrobial activity (zones of inhibition smaller than 10 mm, lasting for 1-2 days). Treatment of catheters with suspensions containing 5 percent (w/v) of norfloxacin or tobramycin resulted in a very rough catheter surface, such that while the antimicrobial activity may be increased, the catheters cannot be used clinically. Only minocycline has been found to be usable at high concentrations which achieve long-term antimicrobial activity without compromising catheter surface smoothness.

### Example

### Rifampin or Minocycline Plus Antiseptics

Methods. Polyurethane catheter segments were treated with one of the following:
5 percent (w/v) chlorhexidine free base and 1 percent (w/v) rifampin in 50 percent (v/v) methanol and 50 percent (v/v) THF with 3 percent (w/v) 60D polyurethane and 1 percent (w/v) 93A polyurethane;
5 percent (w/v) chlorhexidine diacetate and 1 percent (w/v) minocycline in 50 percent (v/v) methanol and 50 percent (v/v) THF with 3 percent (w/v) 60D polyurethane and 1 percent (w/v) 93A polyurethane;
3 percent (w/v) chlorhexidine diacetate and 3 percent (w/v) minocycline in 50 percent (v/v) methanol and 50 percent (v/v) THF with 3 percent (w/v) 60D polyurethane and 1 percent (w/v) 93A polyurethane;
5 percent (w/v) triclosan and 3 percent (w/v) rifampin in 50 percent (v/v) methanol and 50 percent (v/v) THF with 3 percent (w/v) 60D polyurethane and 1 percent (w/v) 93A polyurethane; or
5 percent (w/v) triclosan and 3 percent (w/v) minocycline in 50 percent (v/v) methanol and 50 percent (v/v) THF with 3 percent (w/v) 60D polyurethane and 1 percent (w/v) 93A polyurethane.

Treatment consisted of exposing the catheter segment to treatment solution for 2-5 seconds at room temperature, and then drying the segments in air. Then, 0.5 cm catheter segments were placed vertically on trypticase soy agar (“TSA”) plates seeded with 0.3 ml of 10⁸ cfu/ml of a test culture of either (i) rifampin resistant S. epidermidis; (ii) minocycline passaged S. epidermidis (where the MIC of minocycline did not change); or (iii) S. epidermidis sensitive to minocycline and rifampin. After incubation at 37° C. for 24 hours the zones of inhibition were measured.

Results. The experiments described in the preceding paragraph produced the results shown in Table V.

TABLE V
Zones of Inhibition (mm)

| | | | Minocycline |
|---|---|---|---|
| | | | and |
| | Rifampin | Minocycline | Rifampin |
| CATHETER | Resistant S. | Passaged | Resistant |
| GROUP | epidermidis | S. epidermidis | S. epidermidis |
| 5% Chlorhexidine + 1% | 11 | 21 | 21 |
| Rifampin | | | |
| 5% Chlorhexidine + 1% | 19 | 20 | 20 |
| Minocycline | | | |
| 3% Chlorhexidine + 3% | 20 | 20 | 20 |
| Minocycline | | | |
| 5% Triclosan + 3% | 15 | >25 | >25 |
| Rifampin | | | |
| 5% Triclosan + 3% | 23 | 23 | 24 |
| Minocycline | | | |

Discussion. The foregoing results suggest that minocycline plus antiseptic is a preferred combination for use in medical devices, as bacterial resistance does not appear to develop and sufficient amounts can be incorporated into catheters to provide long-term activity. In addition, minocycline is highly effective against S. epidermidis, one of the major causative organisms of catheter-related infection.

### Example

### Superior Results of Antibiotic Plus Antiseptic

Methods. Polyurethane catheters were treated with one of the following solutions:
(1) 3 percent (w/v) chlorhexidine diacetate (“CHA”) and 0.75 percent (w/v) silver sulfadiazine (“AgSD”) in a solvent consisting of 70 percent (v/v) tetrahydrofuran and 30 percent (v/v) methanol and 3 percent (w/v) 60D polyurethane and 1 percent (w/v) 93A polyurethane;
(2) 3 percent (w/v) minocycline and 3 percent (w/v) rifampin in a solvent consisting of 50 percent (v/v) tetrahydrofuran and 50 percent (v/v) methanol and 3 percent (w/v) 60D polyurethane and 1 percent (w/v) 93A polyurethane;
(3) 3 percent (w/v) chlorhexidine diacetate, 2 percent (w/v) minocycline and 1 percent (w/v) silver sulfadiazine in a solvent consisting of 50 percent (v/v) tetrahydrofuran and 50 percent (v/v) methanol and 3 percent (w/v) 60D polyurethane and 1 percent (w/v) 93A polyurethane;
(4) 5 percent (w/v) triclosan, 1 percent (w/v) silver carbonate (Ag₂CO₃), and 1 percent (w/v) citric acid in a solvent consisting of 70 percent (v/v) tetrahydrofuran and 30 percent (v/v) methanol and 3 percent (w/v) 60D polyurethane and 1 percent (w/v) 93A polyurethane;
(5) 5 percent (w/v) triclosan, 3 percent (w/v) minocycline, and 1 percent (w/v) silver carbonate (Ag₂CO₃) in a solvent consisting of 50 percent (v/v) tetrahydrofuran and 50 percent (v/v) methanol and 3 percent (w/v) 60D polyurethane and 1 percent (w/v) 93A polyurethane;
(6) 6 percent (w/v) chlorhexidine diacetate in a solvent consisting of 70 percent (v/v) tetrahydrofuran and 30 percent (v/v) methanol and 3 percent (w/v) 60D polyurethane and 1 percent (w/v) 93A polyurethane;
(7) 6 percent (w/v) chlorhexidine diacetate and 1 percent (w/v) silver sulfadiazine in a solvent consisting of 70 percent (v/v) tetrahydrofuran and 30 percent (v/v) methanol and 3 percent (w/v) 60D polyurethane and 1 percent (w/v) 93A polyurethane;
(8) 8 percent (w/v) triclosan in a solvent consisting of 70 percent (v/v) tetrahydrofuran and 30 percent (v/v) methanol and 3 percent (w/v) 60D polyurethane and 1 percent (w/v) 93A polyurethane;
(9) 8 percent (w/v) minocycline in a solvent consisting of 50 percent (v/v) tetrahydrofuran and 50 percent (v/v) methanol and 3 percent (w/v) 60D polyurethane and 1 percent (w/v) 93A polyurethane; or
(10) 1 percent (w/v) silver carbonate (Ag₂CO₃) in a solvent consisting of 70 percent (v/v) tetrahydrofuran and 30 percent (v/v) methanol and 3 percent (w/v) 60D polyurethane and 1 percent (w/v) 93A polyurethane.

Catheters were treated by dipping the catheter in the treatment solution for 2-5 seconds and then drying for 24 hours at room temperature. The treated catheters were then cut into segments and tested for their ability to produce zones of inhibition in trypticase soy agar plates seeded with 0.3 mls of cultures of 10⁸ CFU/ml of either S. epidermidis, Pseudomonas aeruginosa, or Enterobacter aerogenes. Zones of inhibition were measured after 24 hours.

Results. The results of the foregoing experiments are depicted in Table VI.

TABLE VI
Zones of Inhibition (mm)

| GROUP | | S. | P. | E. |
|---|---|---|---|---|
| # | TREATMENT | epidermidis | aeruginosa | aerogenes |
| 1 | 3% CHA + 0.75% | 16 | 11 | 11 |
| | AgSD | | | |
| 2 | 3% Minocycline + 3% | 20 | 0 | 15 |
| | Rifampin | | | |
| 3 | 3% CHA + 2% | 22 | 15 | 15 |
| | Minocycline + 1% | | | |
| | AgSD | | | |
| 4 | 5% Triclosan + 1% | 20 | 10 | 12 |
| | Ag₂CO₃ | | | |
| 5 | 5% Triclosan + 1% | 21 | 11 | 12 |
| | Ag₂CO₃ + 1% | | | |
| | Citric Acid | | | |
| 6 | 5% Triclosan + 3% | 25 | 13 | 20 |
| | Minocycline + 1% | | | |
| | Ag₂CO₃ | | | |
| 7 | 6% CHA | 15.5 | 13 | 11 |
| 8 | 6% CHA + 1% | 16 | 13 | 13 |
| | AgSD | | | |
| 9 | 8% Triclosan | 15 | 0 | 11 |
| 10 | 8% Minocycline | 20 | 0 | 9 |
| 11 | 1% Ag₂CO₃ | 10 | 6 | — |

Conclusions. Catheters treated with one or more antiseptics in combination with an antibiotic produce larger zones of inhibition than those treated with single agents.

### Example

### Anti-adherence Effects Of Antibiotic+Antiseptic

Methods. Polyurethane catheters were treated with one of the following solutions:
3 percent (w/v) chlorhexidine diacetate (“CHA”) and 0.75 percent (w/v) silver sulfadiazine (“AgSD”) in 70 percent (v/v) THF and 30 percent (v/v) methanol with 3 percent (w/v) 60D polyurethane and 1 percent (w/v) 93A polyurethane;
3 percent (w/v) minocycline and 3 percent (w/v) rifampin in 50 percent (v/v) methanol and 50 percent (v/v) THF with 3 percent (w/v) 60D polyurethane and 1 percent (w/v) 93A polyurethane;
3 percent (w/v) chlorhexidine diacetate, 2 percent (w/v) minocycline and 0.75 percent (w/v) silver sulfadiazine in 50 percent (v/v) methanol and 50 percent (v/v) THF with 3 percent (w/v) 60D polyurethane and 1 percent (w/v) 93A polyurethane;
5 percent (w/v) triclosan, 1 percent (w/v) silver carbonate (Ag₂CO₃), and 1 percent (w/v) citric acid in 70 percent (v/v) THF and 30 percent (v/v) methanol with 3 percent (w/v) 60D polyurethane and 1 percent (w/v) 93A polyurethane;
5 percent (w/v) triclosan, 3 percent (w/v) minocycline and 1 percent (w/v) silver carbonate (Ag₂CO₃) in 50 percent (v/v) methanol and 50 percent (v/v) THF with 3 percent (w/v) 60D polyurethane and 1 percent (w/v) 93A polyurethane; or

4 cm lengths of catheter, treated as above, were inserted into test tubes containing 5 ml of trypticase soy agar, so that approximately 1 cm of catheter protruded outside the agar. Then, 0.2 ml of a 10⁷ cfu/ml culture of Staphylococcus aureus was applied to the top surface of the agar. The tubes were incubated at 37° C. for seven days. The catheters were then removed and bacterial adherence on the outer surface was determined by rolling the catheter on drug inactivating agar plates, which were incubated for 48 hours at 37° C., after which the bacterial colony counts were determined.

Results. As shown in Table VII, bacterial adherence was found to be lower on catheters impregnated with one or more antiseptics and minocycline.

TABLE VII
Bacterial adherence

| ANTIMICROBIAL TREATMENT | CFU/CM (CATHETER) |
|---|---|
| 3% CHA + 0.75% AgSD | 15 |
| 3% Minocycline + 3% Rifampin | 46 |
| 3% CHA + 3% Minocycline + 0.75% AgSD | 0 |
| 5% Triclosan + 1% Ag₂CO₃ + 1% | 50 |
| Citric Acid | |
| 5% Triclosan + 3% Minocycline + 1% | 0 |
| Ag₂CO₃ | |
| CONTROL | 5 × 10³ |

### Example

### Minocycline Plus Antiseptic Treated Catheters

Methods. Polyurethane catheters were treated with one of the following solutions:
8 percent (w/v) minocycline in 50 percent (v/v) methanol and 50 (v/v) percent THF with 3 percent (w/v) 60D polyurethane and 1 percent (w/v) 93A polyurethane;
8 percent (w/v) triclosan in 70 percent (v/v) THF and 30 percent (v/v) methanol with 3 percent (w/v) 60D polyurethane and 1 percent (w/v) 93A polyurethane; or
5 percent (w/v) triclosan and 3 percent (w/v) minocylcine in 50 percent (v/v) methanol and 50 (v/v) percent THF with 3 percent (w/v) 60D polyurethane and 1 percent (w/v) 93A polyurethane.

The various catheter segments were then tested for their ability to produce zones of inhibition in bacterial lawns produced by seeding trypticase soy agar plates with 0.3 milliliters of cultures of 10⁸ CFU/ml of either Acinetobacter calcoaceticus, Pseudomonas aeruginosa, Enterobacter aerogenes, or Staphylococcus epidermidis. Zones of inhibition were measured after incubating the plates at 37° C. for 24 hours.

Results. As shown in Table VIIIA, the combination of triclosan and minocycline exhibited enhanced activity relative to the concentrations of minocycline or triclosan tested. Since the zones of inhibition against S. epidermidis were turbid and therefore could not be measured very accurately, the cidal activity was checked by subculturing a 1 mm² area from the zone adjacent to the catheter after three daily transfers of the catheter. The results are shown in Table VIIIB. This data shows enhancement of activity of the combination of minocycline and triclosan over and above that of the individual drugs when used alone at the same weight/volume (as opposed to molar) concentration as that of the combination.

TABLE VIIIA
Zones of Inhibition (mm)

| TREAT- | | P. | | S. |
|---|---|---|---|---|
| MENT | Acinetobacter | aeruginosa | Enterobacter | epidermidis |
| 8% | 17 | 0 | 9 | 20 |
| Minocycline | | | | |
| 8% Triclosan | 7 | 0 | 11 | 15 |
| 5% | 17 | 0 | 18 | >25 |
| Triclosan + | | | | |
| 3% | | | | |
| Minocycline | | | | |

TABLE VIIIB
Bacteria Recovered From S. epidermidis Zone of Inhibition

| TREATMENT | CFU/MM² |
|---|---|
| 8% Minocycline | 93 |
| 8% Triclosan | 118 |
| 5% Triclosan + 3% Minocycline | 0 |

The data in Table VIIIB indicate that as regards S. epidermidis, the combination of triclosan and minocycline was more effective than either single agent treated catheter.

### Example

### Activity Against Pseudomonas aeruginosa

Because of the clinical importance of Pseudomonas aeruginosa in catheter-based infections, and because data shown in Table VIIIA failed to show antimicrobial activity against P. aeruginosa, experiments were performed testing the effectiveness of other antiseptics, used in conjunction with minocycline, against this organism.

Polyurethane catheters were treated with one of the following solutions:
2 percent (w/v) bismuth nitrate in 50 percent (v/v) methanol and 50 (v/v) percent THF with 3 percent (w/v) 60D polyurethane and 1 percent (w/v) 93A polyurethane;
5 percent (w/v) minocycline in 50 percent (v/v) methanol and 50 (v/v) percent THF with 3 percent (w/v) 60D polyurethane and 1 percent (w/v) 93A polyurethane;
3 percent (w/v) minocycline and 2 percent (w/v) bismuth nitrate in 50 percent (v/v) methanol and 50 percent (v/v) THF with 3 percent (w/v) 60D polyurethane and 1 percent (w/v) 93A polyurethane;
3 percent (w/v) chlorhexidine diacetate in 70 percent (v/v) THF and 30 percent (v/v) methanol with 3 percent (w/v) 60D polyurethane and 1 percent (w/v) 93A polyurethane;
3 percent (w/v) chlorhexidine diacetate and 3 percent (w/v) minocycline in 50 percent (v/v) methanol and 50 percent (v/v) THF with 3 percent (w/v) 60D polyurethane and 1 percent (w/v) 93A polyurethane;
2 percent (w/v) silver carbonate in 50 percent (v/v) methanol and 50 percent (v/v) THF with 3 percent (w/v) 60D polyurethane and 1 percent (w/v) 93A polyurethane; or
3 percent (w/v) minocycline and 1 percent (w/v) silver carbonate in 50 percent (v/v) methanol and 50 percent (v/v) THF with 3 percent (w/v) 60D polyurethane and 1 percent (w/v) 93A polyurethane.

The treated catheters were then cut into segments and tested for their ability to produce zones of inhibition in a bacterial lawn produced by seeding trypticase soy agar plates with 0.3 mls of a 10⁸ CFU/ml culture of Pseudomonas aeruginosa, placing the catheter segments vertically on the seeded plate, and then incubating for 24 hours at 37° C.

The results are depicted in Table IX.

TABLE IX
Zones of Inhibition

| TREATMENT | ZONE OF INHIBITION (mm) |
|---|---|
| 2% Bismuth Nitrate | 0 |
| 5% Minocycline | 0 |
| 3% Minocycline + 2% Bismuth Nitrate | 17 |
| 3% Chlorhexidine Diacetate | 11 |
| 3% Chlorhexidine Diacetate + 3% | 10.6 |
| Minocycline | |
| 2% Silver Carbonate | 9.0 |
| 3% Minocycline + 1% Silver Carbonate | 9.0 |

In view of the results depicted in Table IX, which demonstrated enhanced antimicrobial effects of bismuth nitrate and minocycline combinations, salts of zinc and cerium in combination with triclosan and/or minocycline were tested as follows.

Polyurethane catheters were treated with one of the following solutions:
5 percent (w/v) triclosan and 3 percent (w/v) minocycline in 50 percent (v/v) methanol and 50 percent (v/v) THF with 3 percent (w/v) 60D polyurethane and 1 percent (w/v) 93A polyurethane;
5 percent (w/v) triclosan, 3 percent (w/v) minocycline and 1 percent (w/v) silver carbonate in 50 percent (v/v) methanol and 50 percent (v/v) THF with 3 percent (w/v) 60D polyurethane and 1 percent (w/v) 93A polyurethane;
5 percent (w/v) triclosan, 3 percent (w/v) minocycline, and 2 percent (w/v) zinc acetate in 50 percent (v/v) methanol and 50 percent (v/v) THF with 3 percent (w/v) 60D polyurethane and 1 percent (w/v) 93A polyurethane;
5 percent (w/v) triclosan, 3 percent (w/v) minocycline, and 2 percent (w/v) bismuth nitrate in 50 percent (v/v) methanol and 50 percent (v/v) THF with 3 percent (w/v) 60D polyurethane and 1 percent (w/v) 93A polyurethane;
1 percent (w/v) silver carbonate in 70 percent (v/v) THF and 30 percent (v/v) methanol with 3 percent (w/v) 60D polyurethane and 1 percent (w/v) 93A polyurethane;
2 percent (w/v) bismuth nitrate in 50 percent (v/v) methanol and 50 percent (v/v) THF with 3 percent (w/v) 60D polyurethane and 1 percent (w/v) 93A polyurethane;
5 percent (w/v) cerium nitrate in 50 percent (v/v) methanol and 50 percent (v/v) THF with 3 percent (w/v) 60D polyurethane and 1 percent (w/v) 93A polyurethane; or
3 percent (w/v) minocycline and 2.4 percent (w/v) cerium nitrate in 50 percent (v/v) methanol and 50 percent (v/v) THF with 3 percent (w/v) 60D polyurethane and 1 percent (w/v) 93A polyurethane.

The salts were used at concentrations which would provide 0.8 percent (w/v) of the metal in each salt.

The catheters were then cut into segments and tested for their ability to produce zones of inhibition in bacterial or yeast lawns produced, respectively, by 0.3 ml of P. aeruginosa or 0.5 ml of Candida albicans, both at culture concentrations of 10⁸ CFU/ml. The following results, set forth in Table X, were obtained.

TABLE X
Zones of Inhibition (mm)

| TREATMENT | P. aeruginosa | C. albicans |
|---|---|---|
| 5% Triclosan + 3% Minocycline | 0 | 0 |
| 5% Triclosan + 3% Minocycline + 1% | 9 | 0 |
| Silver Carbonate | | |
| 5% Triclosan + 3% Minocycline + 2% | 0 | 0 |
| Zinc Acetate | | |
| 5% Triclosan + 3% Minocycline + 2% | 15 | 0 |
| Bismuth Nitrate | | |
| 1% Silver Carbonate | 6 | 0 |
| 2% Bismuth Nitrate | 0 | 0 |
| 5% Cerium Nitrate | 0 | 0 |
| 3% Minocycline + 2.4% Cerium Nitrate | 6 | 0 |

### Example

### Bismuth and Minocycline Combinations

Polyurethane catheters were treated with one of the following solutions:
2 percent (w/v) bismuth nitrate in 50 percent (v/v) methanol and 50 percent (v/v) THF with 3 percent (w/v) 60D polyurethane and 1 percent (w/v) 93A polyurethane;
3 percent (w/v) minocycline in 50 percent (v/v) methanol and 50 percent (v/v) THF with 3 percent (w/v) 60D polyurethane and 1 percent (w/v) 93A polyurethane; or
2 percent (w/v) bismuth nitrate and 3 percent (w/v) minocycline in 50 percent (v/v) methanol and 50 percent (v/v) THF with 3 percent (w/v) 60D polyurethane and 1 percent (w/v) 93A polyurethane.

The various catheter segments were then tested for their ability to produce zones of inhibition in bacterial lawns produced by seeding trypticase soy agar plates with 0.3 mis of cultures of 10⁸ CFU/ml of Staphylococcus epidermidis, Pseudomonas aeruginosa, Acinetobacter calcoaceticus, or Enterobacter aerogenes. Zones of inhibition were measured after culturing the plates at 37° C. for 24 hours. The results are shown in Table XI.

TABLE XI
Zones of Inhibition (mm)

| TREAT- | S. | P. | | |
|---|---|---|---|---|
| MENT | epidermidis | aeruginosa | Acinetobacter | Enterobacter |
| 2% Bismuth | 6 | 0 | 0 | 0 |
| Nitrate | | | | |
| 3% | 23 | 0 | 15 | 11 |
| Minocycline | | | | |
| 2% Bismuth | >25 | 17 | 17 | 18 |
| Nitrate + 3% | | | | |
| Minocycline | | | | |

In order to further improve the antimicrobial spectrum of non-chlorhexidine groups the following combinations were evaluated. Polyurethane catheters were treated with one of the following solutions:
3 percent (w/v) minocycline and 2 percent (w/v) bismuth nitrate in 50 percent (v/v) methanol and 50 percent (v/v) THF with 3 percent (w/v) 60D polyurethane and 1 percent (w/v) 93A polyurethane;
5 percent (w/v) triclosan in 70 percent (v/v) THF and 30 percent (v/v) methanol with 3 percent (w/v) 60D polyurethane and 1 percent (w/v) 93A polyurethane;
3 percent (w/v) minocycline, 2 percent (w/v) bismuth nitrate, and 5 percent (w/v) triclosan in 50 percent (v/v) methanol and 50 percent (v/v) THF with 3 percent (w/v) 60D polyurethane and 1 percent (w/v) 93A polyurethane;
0.5 percent (w/v) benzalkonium chloride (“BZK”) in 70 percent (v/v) THF and 30 percent (v/v) methanol with 3 percent (w/v) 60D polyurethane and 1 percent (w/v) 93A polyurethane; or
3 percent (w/v) minocycline, 2 percent (w/v) bismuth nitrate, and 0.5 percent (w/v) benzalkonium chloride in 50 percent (v/v) methanol and 50 percent (v/v) THF with 3 percent (w/v) 60D polyurethane and 1 percent (w/v) 93A polyurethane.

The ability of treated catheters to produce zones of inhibition was tested as set forth previously in this section, except that the yeast Candida albicans was also added to the test panel, the yeast lawn having been produced using 0.5 ml of a culture of 10⁸ CFU/ml. The results are set forth in Table XII.

TABLE XII
Zones of Inhibition (mm)

| TREATMENT | S. epidermidis | P. aeruginosa | Acinetobacter | Enterobacter | C. albicans |
|---|---|---|---|---|---|
| 3% Minocycline + 2% | >25 | 17 | 17 | 16 | 0 |
| Bismuth | | | | | |
| Nitrate | | | | | |
| 5% Triclosan | 12 | 0 | 5 | 7 | 0 |
| 3% Minocycline + 2% | >25 | 17 | 23 | 19 | 0 |
| Bismuth | | | | | |
| Nitrate + 5% | | | | | |
| Triclosan | | | | | |
| 0.5% BZK | 17 | 0 | 6 | 6 | 10 |
| 3% Minocycline + 2% | >25 | 17 | 20 | 18 | 12 |
| Bismuth | | | | | |
| Nitrate + 0.5% | | | | | |
| BZK | | | | | |

The foregoing results show that the use of triclosan with bismuth salt and minocycline enhanced the antimicrobial activity against Enterobacter and Acinetobacter, both of which are associated with catheter-related infections. Use of BZK with bismuth salt and minocycline improved the antimicrobial spectrum to include C. albicans. Of the groups tested, all except the 5% triclosan treatment groups exhibited good activity against S. epidermidis.

### Example

### BZK, Minocycline and Bismuth Salt

Polyurethane catheters were treated with one of the following solutions:
0.5 percent (w/v) benzalkonium chloride (“BZK”) in 50 percent (v/v) methanol and 50 percent (v/v) THF with 3 percent (w/v) 60D polyurethane and 1 percent (w/v) 93A polyurethane;
0.5 percent (w/v) benzalkonium chloride and 2 percent (w/v) bismuth nitrate in 50 percent (v/v) methanol and 50 percent (v/v) THF with 3 percent (w/v) 60D polyurethane and 1 percent (w/v) 93A polyurethane;
3 percent (w/v) minocycline and 2 percent (w/v) bismuth nitrate in 50 percent (v/v) methanol and 50 percent (v/v) THF with 3 percent (w/v) 60D polyurethane and 1 percent (w/v) 93A polyurethane;
0.5 percent (w/v) benzalkonium chloride and 3 percent (w/v) minocycline in 50 percent (v/v) methanol and 50 percent (v/v) THF with 3 percent (w/v) 60D polyurethane and 1 percent (w/v) 93A polyurethane;
0.5 percent (w/v) benzalkonium chloride, 2 percent (w/v) bismuth nitrate, and 3 percent (w/v) minocycline in 50 percent (v/v) methanol and 50 percent (v/v) THF with 3 percent (w/v) 60D polyurethane and 1 percent (w/v) 93A polyurethane;
5 percent (w/v) triclosan in 70 percent (v/v) THF and 30 percent (v/v) methanol with 3 percent (w/v) 60D polyurethane and 1 percent (w/v) 93A polyurethane;
3 percent (w/v) minocycline, 2 percent (w/v) bismuth nitrate and 5 percent (w/v) triclosan in 50 percent (v/v) methanol and 50 percent (v/v) THF with 3 percent (w/v) 60D polyurethane and 1 percent (w/v) 93A polyurethane; and, as CONTROL, untreated catheters.

The treated catheters were then dried, cut into segments and used to produce zones of inhibition on lawns of S. epidermidis produced by seeding trypticase soy agar plates with 0.3 mis of a 10⁸ CFU/ml culture, and then incubating the seeded plates, with the catheter segments vertically placed, for 24 hours at 37° C.

The zones of inhibition were found to be too large to be accurately measurable, so that a one square millimeter area from within the zone of inhibition was subcultured on an antibiotic free plate. The results are shown in Table XIII. Use of triclosan or BZK was found to enhance the cidal activity of bismuth salt and minocycline.

TABLE XIII
RECOVERED BACTERIA FROM ZONES OF INHIBITION

| TREATMENT | CFU/mm² |
|---|---|
| 0.5% BZK | 100 |
| 0.5% BZK + 2% Bismuth nitrate | 90 |
| 3% Minocycline + 2% Bismuth nitrate | 200 |
| 0.5% BZK + 3% Minocycline | 100 |
| 0.5% BZK + 2% Bismuth nitrate + 3% | 2.5 |
| Minocycline | |
| 5% Triclosan | 150 |
| 3% Minocycline + 2% Bismuth nitrate + 5% | 65 |
| Triclosan | |
| CONTROL | 10⁴ |

### Example

### Antimicrobial/Antiseptic Combinations

Polyurethane catheters were treated with one of the following solutions:
2 percent (w/v) bismuth nitrate in 50 percent (v/v) methanol and 50 percent (v/v) THF with 3 percent (w/v) 60D polyurethane and 1 percent (w/v) 93A polyurethane;
5 percent (w/v) triclosan and 3 percent (w/v) minocycline in 50 percent (v/v) methanol and 50 percent (v/v) THF with 3 percent (w/v) 60D polyurethane and 1 percent (w/v) 93A polyurethane;
2 percent (w/v) bismuth nitrate and 3 percent (w/v) minocycline in 50 percent (v/v) methanol and 50 percent (v/v) THF with 3 percent (w/v) 60D polyurethane and 1 percent (w/v) 93A polyurethane;
2 percent (w/v) bismuth nitrate and 3 percent (w/v) rifampin in 50 percent (v/v) methanol and 50 percent (v/v) THF with 3 percent (w/v) 60D polyurethane and 1 percent (w/v) 93A polyurethane;
2 percent (w/v) bismuth nitrate, 3 percent (w/v) minocycline, and 5 percent (w/v) triclosan with 3 percent (w/v) 60D polyurethane and 1 percent (w/v) 93A polyurethane;
2 percent (w/v) bismuth nitrate, 3 percent (w/v) rifampin, and 5 percent (w/v) triclosan in 50 percent (v/v) methanol and 50 percent (v/v) THF with 3 percent (w/v) 60D polyurethane and 1 percent (w/v) 93A polyurethane;
2 percent (w/v) bismuth nitrate, 3 percent (w/v) minocycline, and 0.5 percent (w/v) benzalkonium chloride in 50 percent (v/v) methanol and 50 percent (v/v) THF with 3 percent (w/v) 60D polyurethane and 1 percent (w/v) 93A polyurethane;
5 percent (w/v) triclosan and 2 percent (w/v) bismuth nitrate in 50 percent (v/v) methanol and 50 percent (v/v) THF with 3 percent (w/v) 60D polyurethane and 1 percent (w/v) 93A polyurethane;
2 percent (w/v) bismuth nitrate and 0.5 percent (w/v) benzalkonium chloride in 50 percent (v/v) methanol and 50 percent (v/v) THF with 3 percent (w/v) 60D polyurethane and 1 percent (w/v) 93A polyurethane;
5 percent (w/v) triclosan, 3 percent (w/v) minocycline, and 1 percent (w/v) silver carbonate in 50 percent (v/v) methanol and 50 percent (v/v) THF with 3 percent (w/v) 60D polyurethane and 1 percent (w/v) 93A polyurethane;
3 percent (w/v) minocycline and 1 percent (w/v) silver carbonate in 50 percent (v/v) methanol and 50 percent (v/v) THF with 3 percent (w/v) 60D polyurethane and 1 percent (w/v) 93A polyurethane;
5 percent (w/v) minocycline in 50 percent (v/v) methanol and 50 percent (v/v) THF with 3 percent (w/v) 60D polyurethane and 1 percent (w/v) 93A polyurethane;
5 percent (w/v) rifampin in 50 percent (v/v) methanol and 50 percent (v/v) THF with 3 percent (w/v) 60D polyurethane and 1 percent (w/v) 93A polyurethane; or
0.5 percent (w/v) benzalkonium chloride in 50 percent (v/v) methanol and 50 percent (v/v) THF with 3 percent (w/v) 60D polyurethane and 1 percent (w/v) 93A polyurethane.

The various catheter segments were then tested for their ability to produce zones of inhibition in bacterial lawns produced by seeding trypticase soy agar plates with 0.3 mls of cultures of 10⁸ CFU/ml of Staphylococcus epidermidis, Pseudomonas aeruginosa, Acinetobacter calcoaceticus, Enterobacter aerogenes, or Candida albicans. Zones of inhibition were measured after culturing the plates at 37° C. for 24 hours. The results are shown in Table XIV.

The data indicates that the combination of bismuth nitrate and an antibiotic, either minocycline or rifampin, showed enhanced activity against P. aeruginosa. The combination of minocylcine and bismuth salt appeared to have superior activity, in this regard, than the combination of rifampin and bismuth salt. The combination of bismuth nitrate and minocycline also exhibited enhanced activity against Acinetobacter and Enterobacter bacteria. The addition of benzalkonium chloride to this combination increased antimicrobial activity against C. albicans and Acinetobacter. Further, the combination of benzalkonium chloride, minocycline, and bismuth salt was found to exhibit broad spectrum antimicrobial activity.

TABLE XIV
Zones of Inhibition (mm)

| TREATMENT | P. aeruginosa | Acinetobacter | S. epidermidis | C. albicans | Enterobacter |
|---|---|---|---|---|---|
| 2% Bismuth | 0 | 0 | 0 | 0 | 0 |
| nitrate | | | | | |
| 5% Triclosan + 3% | 0 | 15 | 20 | 0 | 12.5 |
| Minocycline | | | | | |
| 2% Bismuth | 15 | 17 | >25 | 0 | 17 |
| nitrate + 3% | | | | | |
| Minocycline | | | | | |
| 2% Bismuth | 11 | 18 | >25 | 0 | — |
| nitrate + 3% | | | | | |
| Rifampin | | | | | |
| 2% Bismuth | 17 | 21 | >25 | 0 | 17 |
| nitrate + 3% | | | | | |
| Minocycline + 5% | | | | | |
| Triclosan | | | | | |
| 2% Bismuth | 9 | 16 | >25 | 0 | — |
| nitrate + 3% | | | | | |
| Rifampin + 5% | | | | | |
| Triclosan | | | | | |
| 2% Bismuth | 17 | 20 | 25 | 12 | 16 |
| nitrate + 3% | | | | | |
| Minocycline + 0.5% | | | | | |
| BZK | | | | | |
| 5% Triclosan + 2% | 0 | 5 | 17 | 0 | — |
| Bismuth nitrate | | | | | |
| 2% Bismuth | 0 | 7 | 15 | 10 | — |
| nitrate + 0.5% BZK | | | | | |
| 5% Triclosan + 3% | 6 | 15 | 21 | 0 | 18 |
| Minocycline + 1% | | | | | |
| Ag₂CO₃ | | | | | |
| 3% Minocycline + 1% | 8 | 14 | 24 | 0 | — |
| Ag₂CO₃ | | | | | |
| 5% Minocycline | 0 | 15 | 20 | 0 | 16 |
| 5% Rifampin | 8 | 15 | 20 | 0 | 16 |
| 0.5% BZK | 0 | 6.5 | 17 | 10 | 6 |

### Example

### Combinations of Antibiotics and Bismuth Nitrate

Polyurethane catheters were treated with one of the following solutions:
5 percent (w/v) rifampin in 50 percent (v/v) methanol and 50 percent (v/v) THF with 3 percent (w/v) 60D polyurethane and 1 percent (w/v) 93A polyurethane;
5 percent (w/v) rifampin and 2 percent (w/v)bismuth nitrate in 50 percent (v/v) methanol and 50 percent (v/v) THF with 3 percent (w/v) 60D polyurethane and 1 percent (w/v) 93A polyurethane;
5 percent (w/v) gentamycin in 50 percent (v/v) methanol and 50 percent (v/v) THF with 3 percent (w/v) 60D polyurethane and 1 percent (w/v) 93A polyurethane;
5 percent (w/v) gentamycin and 2 percent (w/v) bismuth nitrate in 50 percent (v/v) methanol and 50 percent (v/v) THF with 3 percent (w/v) 60D polyurethane and 1 percent (w/v) 93A polyurethane;
5 percent (w/v) tobramycin in 50 percent (v/v) methanol and 50 percent (v/v) THF with 3 percent (w/v) 60D polyurethane and 1 percent (w/v) 93A polyurethane;
5 percent (w/v) tobramycin and 2 percent (w/v) bismuth nitrate in 50 percent (v/v) methanol and 50 percent (v/v) THF with 3 percent (w/v) 60D polyurethane and 1 percent (w/v) 93A polyurethane;
5 percent (w/v) ceftazidine in 50 percent (v/v) methanol and 50 percent (v/v) THF with 3 percent (w/v) 60D polyurethane and 1 percent (w/v) 93A polyurethane;
5 percent (w/v) ceftazidine and 2 percent (w/v) bismuth nitrate in 50 percent (v/v) methanol and 50 percent (v/v) THF with 3 percent (w/v) 60D polyurethane and 1 percent (w/v) 93A polyurethane;
5 percent (w/v) dicloxacillin in 50 percent (v/v) methanol and 50 percent (v/v) THF with 3 percent (w/v) 60D polyurethane and 1 percent (w/v) 93A polyurethane;
5 percent (w/v) dicloxacillin and 2 percent (w/v) bismuth nitrate in 50 percent (v/v) methanol and 50 percent (v/v) THF with 3 percent (w/v) 60D polyurethane and 1 percent (w/v) 93A polyurethane;
5 percent (w/v) norfloxacin in 50 percent (v/v) methanol and 50 percent (v/v) THF with 3 percent (w/v) 60D polyurethane and 1 percent (w/v) 93A polyurethane;
5 percent (w/v) norfloxacin and 2 percent (w/v) bismuth nitrate in 50 percent (v/v) methanol and 50 percent (v/v) THF with 3 percent (w/v) 60D polyurethane and 1 percent (w/v) 93A polyurethane;
5 percent (w/v) minocycline in 50 percent (v/v) methanol and 50 percent (v/v) THF with 3 percent (w/v) 60D polyurethane and 1 percent (w/v) 93A polyurethane;
5 percent (w/v) minocycline and 2 percent (w/v) bismuth nitrate in 50 percent (v/v) methanol and 50 percent (v/v) THF with 3 percent (w/v) 60D polyurethane and 1 percent (w/v) 93A polyurethane;
5 percent (w/v) bacitracin in 50 percent (v/v) methanol and 50 percent (v/v) THF with 3 percent (w/v) 60D polyurethane and 1 percent (w/v) 93A polyurethane;
5 percent (w/v) bacitracin and 2 percent (w/v) bismuth nitrate in 50 percent (v/v) methanol and 50 percent (v/v) THF with 3 percent (w/v) 60D polyurethane and 1 percent (w/v) 93A polyurethane;
5 percent (w/v) miconazole in 50 percent (v/v) methanol and 50 percent (v/v) THF with 3 percent (w/v) 60D polyurethane and 1 percent (w/v) 93A polyurethane;
5 percent (w/v) miconazole and 2 percent (w/v) bismuth nitrate in 50 percent (v/v) methanol and 50 percent (v/v) THF with 3 percent (w/v) 60D polyurethane and 1 percent (w/v) 93A polyurethane; or 3 percent (w/v) bismuth nitrate in 50 percent (v/v) methanol and 50 percent (v/v) THF with 3 percent (w/v) 60D polyurethane and 1 percent (w/v) 93A polyurethane.

The various catheter segments were then tested for their ability to produce zones of inhibition in bacterial lawns produced by seeding trypticase soy agar plates with cultures of 0.3 ml of a culture of 10⁸ CFU/ml of Pseudomonas aeruginosa. Catheter segments were placed vertically on the seeded plates, which were then incubated at 37° C. for 24 hours, after which the zones of inhibition of bacterial growth were measured.

The results are shown in Table XV.

TABLE XV
Zones of Inhibition Against Pseudomonas aeruginosa(mm)

| | | TREATMENT: |
|---|---|---|
| | TREATMENT*: | 5% ANTIBIOTIC + 2% |
| ANTIBIOTIC | 5% ANTIBIOTIC | BISMUTH NITRATE |
| Rifampin | 8.0 | 11 |
| Gentamycin | 22 | 21 |
| Tobramycin | 27 | 23 |
| Ceftazidine | 28 | 27 |
| Dicloxacillin | 0 | 0 |
| Norfloxacin | 25 | 24 |
| Minocycline | 0 | 15 |
| Bacitracin | 0 | 0 |
| Miconazole | 0 | 0 |
| 3% Bismuth Nitrate | 0 | |

*except for 3% Bismuth Nitrate treated catheter

### Example

### Various Bismuth Salt/Minocycline Combinations

Polyurethane catheters were treated with one of the following solutions:
2 percent (w/v) bismuth nitrate in 50 percent (v/v) methanol and 50 percent (v/v) THF with 3 percent (w/v) 60D polyurethane and 1 percent (w/v) 93A polyurethane;
2 percent (w/v) bismuth nitrate and 3 percent (w/v) minocycline in 50 percent (v/v) methanol and 50 percent (v/v) THF with 3 percent (w/v) 60D polyurethane and 1 percent (w/v) 93A polyurethane;
2 percent (w/v) bismuth acetate in 50 percent (v/v) methanol and 50 percent (v/v) THF with 3 percent (w/v) 60D polyurethane and 1 percent (w/v) 93A polyurethane;
2 percent (w/v) bismuth acetate and 3 percent (w/v) minocycline in 50 percent (v/v) methanol and 50 percent (v/v) THF with 3 percent (w/v) 60D polyurethane and 1 percent (w/v) 93A polyurethane;
2 percent (w/v) bismuth citrate in 50 percent (v/v) methanol and 50 percent (v/v) THF with 3 percent (w/v) 60D polyurethane and 1 percent (w/v) 93A polyurethane;
2 percent (w/v) bismuth citrate and 3 percent (w/v) minocycline in 50 percent (y/v) methanol and 50 percent (v/v) THF with 3 percent (w/v) 60D polyurethane and 1 percent (w/v) 93A polyurethane;
2 percent (w/v) bismuth salicylate in 50 percent (v/v) methanol and 50 percent (v/v) THF with 3 percent (w/v) 60D polyurethane and 1 percent (w/v) 93A polyurethane;
2 percent (w/v) bismuth salicylate and 3 percent (w/v) minocycline in 50 percent (v/v) methanol and 50 percent (v/v) THF with 3 percent (w/v) 60D polyurethane and 1 percent (w/v) 93A polyurethane;
2 percent (w/v) bismuth borate in 50 percent (v/v) methanol and 50 percent (v/v) THF with 3 percent (w/v) 60D polyurethane and 1 percent (w/v) 93A polyurethane;
2 percent (w/v) bismuth borate and 3 percent (w/v) minocycline in 50 percent (v/v) methanol and 50 percent (v/v) THF with 3 percent (w/v) 60D polyurethane and 1 percent (w/v) 93A polyurethane;
2 percent (w/v) bismuth mandelate in 50 percent (v/v) methanol and 50 percent (v/v) THF with 3 percent (w/v) 60D polyurethane and 1 percent (w/v) 93A polyurethane;
2 percent (w/v) bismuth mandelate and 3 percent (w/v) minocycline in 50 percent (v/v) methanol and 50 percent (v/v) THF with 3 percent (w/v) 60D polyurethane and 1 percent (w/v) 93A polyurethane;
2 percent (w/v) bismuth palmitate in 50 percent (v/v) methanol and 50 percent (v/v) THF with 3 percent (w/v) 60D polyurethane and 1 percent (w/v) 93A polyurethane;
2 percent (w/v) bismuth palmitate and 3 percent (w/v) minocycline in 50 percent (v/v) methanol and 50 percent (v/v) THF with 3 percent (w/v) 60D polyurethane and 1 percent (w/v) 93A polyurethane;
2 percent (w/v) bismuth benzoate in 50 percent (v/v) methanol and 50 percent (v/v) THF with 3 percent (w/v) 60D polyurethane and 1 percent (w/v) 93A polyurethane;
2 percent (w/v) bismuth benzoate and 3 percent (w/v) minocycline in 50 percent (v/v) methanol and 50 percent (v/v) THF with 3 percent (w/v) 60D polyurethane and 1 percent (w/v) 93A polyurethane;
2 percent (w/v) bismuth sulfadiazine in 50 percent (v/v) methanol and 50 percent (v/v) THF with 3 percent (w/v) 60D polyurethane and 1 percent (w/v) 93A polyurethane;
2 percent (w/v) bismuth sulfadiazine and 3 percent (w/v) minocycline in 50 percent (v/v) methanol and 50 percent (v/v) THF with 3 percent (w/v) 60D polyurethane and 1 percent (w/v) 93A polyurethane; or 5 percent (w/v) minocycline in 50 percent (v/v) methanol and 50 percent (v/v) THF with 3 percent (w/v) 60D polyurethane and 1 percent (w/v) 93A polyurethane.

The various catheter segments were then tested for their ability to produce zones of inhibition in bacterial lawns produced by seeding trypticase soy agar plates with 0.3 ml of a culture of 10⁸ CFU/ml of Pseudomonas aeruginosa. Catheter segments were placed vertically on the seeded plates, which were then incubated at 37° C. for 24 hours, after which the zones of inhibition of bacterial growth were measured.

The results are shown in Table XVI, and demonstrate that minocycline enhances the anti-Pseudomonas activity of bismuth salts.

TABLE XVI
Zones of Inhibition Against Pseudomonas aeruginosa (mm)

| | | 2% Bismuth Salt + 3% |
|---|---|---|
| Bismuth Salt | 2% Bismuth Salt | Minocycline |
| Bismuth Nitrate | 0 | 15 |
| Bismuth acetate | 0 | 17 |
| Bismuth citrate | 0 | 17 |
| Bismuth salicylate | 0 | 17 |
| Bismuth borate | 0 | 13 |
| Bismuth mandelate | 0 | 18.5 |
| Bismuth palmitate | 0 | 18.5 |
| Bismuth benzoate | 0 | 18 |
| Bismuth sulfadiazine | 6.5 | 15.5 |

### Example

### Broad-spectrum Antimicrobial Activity

Polyurethane catheters were treated with one of the following solutions:
5 percent (w/v) triclosan and 3 percent (w/v) minocycline in 50 percent (v/v) methanol and 50 percent (v/v) THF with 3 percent (w/v) 60D polyurethane and 1 percent (w/v) 93A polyurethane;
5 percent (w/v) triclosan, 3 percent (w/v) minocycline, and 1 percent (w/v) silver carbonate in 50 percent (v/v) methanol and 50 percent (v/v) THF with 3 percent (w/v) 60D polyurethane and 1 percent (w/v) 93A polyurethane;
5 percent (w/v) triclosan, 3 percent (w/v) minocycline, and 0.5% (w/v) benzalkonium chloride (“BZK”) in 50 percent (v/v) methanol and 50 percent (v/v) THF with 3 percent (w/v) 60D polyurethane and 1 percent (w/v) 93A polyurethane;
3 percent (w/v) chlorhexidine free base and 3 percent (w/v) minocycline in 50 percent (v/v) methanol and 50 percent (v/v) THF with 3 percent (w/v) 60D polyurethane and 1 percent (w/v) 93A polyurethane;
3 percent (w/v) chlorhexidine free base, 3 percent (w/v) minocycline and 1 percent (w/v) silver carbonate in 50 percent (v/v) methanol and 50 percent (v/v) THF with 3 percent (w/v) 60D polyurethane and 1 percent (w/v) 93A polyurethane;
3 percent (w/v) chlorhexidine free base, 2 percent (w/v) triclosan and 2 percent (w/v) minocycline in 50 percent (v/v) methanol and 50 percent (v/v) THF with 3 percent (w/v) 60D polyurethane and 1 percent (w/v) 93A polyurethane;
0.5 percent (w/v) benzalkonium chloride, 3 percent (w/v) minocycline and 2 percent (w/v) bismuth nitrate in 50 percent (v/v) methanol and 50 percent (v/v) THF with 3 percent (w/v) 60D polyurethane and 1 percent (w/v) 93A polyurethane; or
6 percent (w/v) chlorhexidine diacetate in 70 percent (v/v) THF and 30 percent (v/v) methanol with 3 percent (w/v) 60D polyurethane and 1 percent (w/v) 93A polyurethane.

The various catheter segments were then tested for their ability to produce zones of inhibition in bacterial lawns produced by seeding trypticase soy agar plates with 0.3 mls of cultures of 10⁸ CFU/ml of Pseudomonas aeruginosa, Acinetobacter calcoaceticus, Staphylococcus epidermidis, or Candida albicans. Zones of inhibition were measured after culturing the plates at 37° C. for 24 hours. The results are shown in Table XVII.

TABLE XVII
Zones of Inhibition (mm)

| | P. | | S. | C. |
|---|---|---|---|---|
| TREATMENT | aeruginosa | Acinetobacter | epidermidis | albicans |
| 5% Triclosan + | 0 | 15 | 22 | 0 |
| 3% Minocycline | | | | |
| 5% Triclosan + | 6 | 15 | 22 | 0 |
| 3% | | | | |
| Minocycline + | | | | |
| 1% Silver | | | | |
| Carbonate | | | | |
| 5% Triclosan + | 0 | 15 | 21 | 7 |
| 3% | | | | |
| Minocycline + | | | | |
| 0.5% BZK | | | | |
| 3% CHX free | 11 | 12 | 22 | 8 |
| base + | | | | |
| 3% Minocycline | | | | |
| 3% CHX free | 12 | 12 | 22 | 10 |
| base + 3% | | | | |
| Minocycline + | | | | |
| 1% Silver | | | | |
| Carbonate | | | | |
| 3% CHX free | 11 | 13 | 22 | 8 |
| base + | | | | |
| 2% Triclosan + | | | | |
| 2% Minocycline | | | | |
| 0.5% BZK + | 15 | 20 | 25 | 12 |
| 3% | | | | |
| Minocycline + | | | | |
| 2% Bismuth | | | | |
| Nitrate | | | | |
| 6% CHA | 10 | 11 | 15 | 9 |

### Example

### Minocycline Plus Chlorhexidine Treated Catheters

Methods. Polyurethane catheters were treated with one of the following solutions:
3.5 percent chlorhexidine diacetate (CHA) and 0.75 percent silver sulfadiazine (AgSD) and 3 percent 93A polyurethane (93A) and 1 percent 60D polyurethane (60D) in 70 percent (v/v) tetrahydrofuran (THF) and 30 percent (v/v) methanol (MeOH) at room temperature for 2 to 5 seconds;
3 percent chlorhexidine free base (CHX) and 1 percent minocycline (M) and 3.5 percent 60D polyurethane in 50 percent THF at room temperature at 2 to 5 seconds; or
were purchased catheters impregnated with minocycline (M) and rifampin (R) (commercially available from Cook Critical Care) that contain 0.5 mg of M and 0.5 mg of R.

The various catheters were then tested for their ability to produce zones of inhibition in bacterial or yeast lawns produced by seeding trypticase soy ager plates with 0.3 ml of either S. epidermidis or A. calcoaceticus, or 0.5 ml of C. albicans, all at culture concentrations of 1×10⁸ cfu/ml. 0.5 cm segments of the test catheters were then embedded vertically in the plates. The zones of inhibition were measured after incubating the plates at 37° C. for 24 hours.

Results. The drug levels of chlorhexidine and minocycline in the catheters produced above are shown in Table XVIII below.

TABLE XVIII
Drug Levels (μg/cm)

| Catheter Group | Chlorhexidine | Minocycline |
|---|---|---|
| 3.5% CHA + 0.75% | 602 | — |
| AgSD + 3% | | |
| 93A + 1% | | |
| 60D + 70% | | |
| THF* + 30% | | |
| MeOH* | | |
| MR | 500 | 500 |
| 3% CHX + 1% | 366 | 161 |
| M + 3.5% | | |
| 60D + 1% | | |
| 93A + 50% | | |
| MeOH* + 50% | | |
| THF* | | |

*= v/v

The experiments described in the preceding paragraphs produced the results shown in Tables XIX and XX.

TABLE XIX
Zones of inhibition of S. epidermidis (mm)

| Catheter | Day | | | | | | |
|---|---|---|---|---|---|---|---|
| Group | 1 | 2 | 3 | 4 | 5 | 6 | 7 |
| 3.5% CHA + 0.75% | 15 | 12 | 11 | 10 | 9 | 9 | 9 |
| AgSD + 3% | | | | | | | |
| 93A + 1% | | | | | | | |
| 60D + 70% | | | | | | | |
| THF* + 30% | | | | | | | |
| MeOH* | | | | | | | |
| MR | 22 | 20 | 18 | 18 | 17 | 17 | 17 |
| 3% CHX + 1% | 21.3 | 20.6 | 21 | 21 | 20 | 19.5 | 20 |
| M + 3.5% | | | | | | | |
| 60D + 1% | | | | | | | |
| 93A + 50% | | | | | | | |
| MeOH* + 50% | | | | | | | |
| THF* | | | | | | | |

*= v/v

TABLE XX
Zones of Inhibition of A. calcoaceticus (mm)

| | Day | | | | | | |
|---|---|---|---|---|---|---|---|
| Catheter Group | 1 | 2 | 3 | 4 | 5 | 6 | 7 |
| 3.5% CHA + 0.75% | 9.2 | 6.8 | 6.3 | 4.3 | 6.7 | 4 | 3.8 |
| AgSD + 3% | | | | | | | |
| 93A + 1% | | | | | | | |
| 60D + 70% | | | | | | | |
| THF* + 30% | | | | | | | |
| MeOH* | | | | | | | |
| MR | ND | | | | | | |
| 3% CHX + 1% | 10.8 | 10 | 10.1 | 10 | 10 | 9.7 | 9.3 |
| M + 3.5% | | | | | | | |
| 60D + 1% | | | | | | | |
| 93A + 50% | | | | | | | |
| MeOH* + 50% | | | | | | | |
| THF* | | | | | | | |

ND—not done
*= v/v

The foregoing results demonstrate that catheters treated with minocycline plus chlorhexidine exhibited enhanced activity for a longer period of time against S. epidermidis and A. calcoaceticus when compared to the other groups of catheters.

### Example

### Anti-adherence Effects of Minocycline Plus Chlorhexidine Treated Catheters Against S. Aureus

Methods. The efficacy of minocycline plus chlorhexidine treated catheters was tested using an ager tract model in vitro and a rat subcutaneous model in vivo for determining bacterial adherence of S. aureus.

Polyurethane catheter segments were treated with one of the following solutions:
3.5 percent chlorhexidine diacetate (CHA) and 0.75 percent silver sulfadiazine (AgSD) and 3 percent 93A polyurethane (93A) and 1 percent 60D polyurethane (60D) in 70 percent (v/v) tetrahydrofuran (THF) and 30 percent (v/v) methanol (MeOH) at room temperature for 2 to 5 seconds;
3 percent chlorhexidine free base (CHX) and 1 percent minocycline (M) and 3.5 percent 60D polyurethane in 50 percent THF at room temperature at 2 to 5 seconds; or
were purchased catheters impregnated with minocycline (M) and rifampin (R) (commercially available from Cook Critical Care) that contain 0.5 mg of M and 0.5 mg of R.

Culture tubes containing 12.5 ml of culture medium (0.5% agar+0.03% TSB+20% BAS+0.5% Parmalat) were prepared. 4 cm segments of the various catheters were implanted vertically in the soft agar medium so that 0.5 cm of the catheter on one end would project out of the medium. The catheters were transferred to fresh medium at desired intervals to simulate in vivo drug clearance and infected 24 hours later with 20 μl of a S. aureus culture containing 1×10⁷ cfu/ml at the insertion site. The tubes were then incubated at 37° C. for 7 days. Bacterial adherence was then determined by removing the catheters from the medium, rinsing the catheters twice, blotting them dry, cutting 1.0 cm segments off both ends, cutting a 2 cm segment from the middle of the segments, suspending the segments in 4 ml LTSB, and sonicating them for 20 minutes. Thereafter, 0.5 ml of the LTSB was seeded on TSA plates or DE plates.

In vivo model. For the in vivo infection studies, catheters were processed similarly to the catheters in the agar tract in vitro study above, except that the catheters were implanted into the subcutaneous pouches of rats and were infected at desired time intervals with 25 μl of 1×10⁸ cfu/ml of S. aureus culture. Bacterial adherence was determined similarly to the catheters in the agar tract in vitro study above, except that the catheters were removed from the subcutaneous pouches 7 days after infection.

Results. The results, using the in vitro agar tract model and the in vivo rat subcutaneous model, of the experiments described in the preceding paragraphs is shown in Table XXI.

TABLE XXI
S. Aureus adherence (cfu/cm)

| | In vitro model | | | In vivo model | |
|---|---|---|---|---|---|
| | Day of infection | | | Day of infection | |
| Catheter Group | 14 | 21 | 44 | 14 | 21 |
| 3.5% CHA + 0.75% | 0 | 2 | 265 | 0 | 31 |
| AgSD + 3% | | | | | |
| 93A + 1% | | | | | |
| 60D + 70% | | | | | |
| THF* + 30% | | | | | |
| MeOH* | | | | | |
| MR | 0 | 0 | 62 | 0 | ND |
| 3% CHX + 1% | 3 | 0 | 0 | 0 | 0 |
| M + 3.5% | | | | | |
| 60D + 1% | | | | | |
| 93A + 50% | | | | | |
| MeOH* + 50% | | | | | |
| THF* | | | | | |
| Control | 10³ | 10³ | 10³ | 10³ | 10³ |

*= v/v

The foregoing results demonstrate that although all groups of catheters when infected on the 14^{th} and 21^{st} day post implantation were equally effective both in the in vitro and in vivo models, the minocycline plus chlorhexidine catheters had significantly lower colonization over the other groups of catheters on the 44^{th} day.

### Example

### Anti-adherence Effects of Minocycline Plus Chlorhexidine Treated Catheters Against P. Aeruginosa

Methods. Polyurethane catheters were treated with one of the solutions as described in the prior example using the in vitro model of determining bacterial adherence as described in the prior example.

The various catheters were tested by implanting catheter segments in the test medium as described in the prior example, except 5 days later the medium was changed and on day 6 the insertion site was inoculated with 20 μl of a 1×10³ cfu/ml P. aeruginosa suspension. After a 7 day incubation, bacterial adherence was determined as described in the prior Example.

Results. As shown in Table XXII, bacterial adherence of P. aeruginosa was found to be lower on catheters treated with minocycline plus chlorhexidine.

TABLE XXII
P. aeruginosa adherence (cfu/cm)

| Catheter Groups | Day of Infection 6 |
|---|---|
| Control | 1.0 × 10³ |
| 3.5% CHA + 0.75% | 1.8 × 10³ |
| AgSD + 3% | |
| 93A + 1% | |
| 60D + 70% | |
| THF* + 30% | |
| MeOH* | |
| MR | 1.0 × 10³ |
| 3% CHX + 1% | 5.8 × 10² |
| M + 3.5% | |
| 60D + 1% | |
| 93A + 50% | |
| MeOH* + 50% | |
| THF* | |

*= v/v

### Example

### Anti-Adherence of Minocycline Plus Chlorhexidine Treated Catheters Against C. Albicans

Methods. Polyurethane catheters were treated with one of the solutions as described in the prior example against P. aeroginosa using the agar tract in vitro model, except the test medium was supplement with 0.5% galactose and 0.1 mM CaCl₂ to enhance the adherence of the yeast C. albicans. Catheters were infected on day 0 at immediately after implantation, at 4 hours and at 4 days past implantation after changing the medium with 20 μl of a 1×10⁷ cfu/ml suspension of C. albicans. Adherence was tested 7 days later as described in the prior example against P. aeruginosa.

Results. As shown in Table XXIII adherence of C. albicans was found to be lower on catheters treated with minocycline plus chlorhexidine than the other catheter groups.

TABLE XXIII
C. albicans adherence (cfu/cm)

| | Day of Infection | | |
|---|---|---|---|
| Catheter Groups | 0 | 4 hour | 4 |
| 3.5% CHA + 0.75% | 3 | 7 | 1.5 × 10² |
| AgSD + 3% | | | |
| 93A + 1% | | | |
| 60D + 70% | | | |
| THF* + 30% | | | |
| MeOH* | | | |
| MR | 1 × 10³ | 1 × 10³ | 1 × 10³ |
| 3% CHX + 1% | 0 | 1 | 24 |
| M + 3.5% | | | |
| 60D + 1% | | | |
| 93A + 50% | | | |
| MeOH* + 50% | | | |
| THF* | | | |
| Control | 5 × 10² | 6 × 10² | 5 × 10² |

*= v/v

### Example

### Long-Term Anti-adherence Effects of Minocycline Plus Chlorhexidine Treated Catheters Against S. Aureus

Methods. In order to simulate conditions in which catheters are surrounded by large amounts of fluid for a long period of time, polyurethane catheters were treated with one of the solutions as described in the prior example against S. aureus using the in vivo model, except 4 cm segments of the test catheters were implanted in peritoneal fluid in the ventral side of the rats and removed after 27 days. These segments were than implanted in agar tract medium and infected with S. aureus 24 hours later. After 7 days of infection, the catheters were processed for adherence in described in the prior example against S. aureus.

Results. The experiments described in the preceding paragraphs produced the results shown in Table XXIV.

TABLE XXIV
S. aureus adherence (cfu/cm)

| | Day of Infection |
|---|---|
| Catheter Group | 27 |
| Control | 1.0 × 10³ |
| 3.5% CHA + 0.75% | 4.8 × 10³ |
| AgSD + 3% | |
| 93A + 1% | |
| 60D + 70% | |
| THF* + 30% | |
| MeOH* | |
| 3% CHX + 1% | 3.1 × 10² |
| M + 3.5% | |
| 60D + 1% | |
| 93A + 50% | |
| MeOH* + 50% | |
| THF* | |

*= v/v

The foregoing results demonstrate that the adherence of S. aureus was found to be 1 log lower of colonies on catheters treated with minocycline plus chlorhexidine than on the other catheter groups.

### Example

### Luminal Antimicrobial Efficacy of Minocycline Plus Chlorhexidine Treated Catheters

Methods. Polyurethane catheters were treated on both the luminal and external surfaces with one of the following:
1.2 percent CHA and 1.2 percent CHX in 80 percent (v/v) MeOH and 20 percent (v/v) THF;
2 percent CHX and 1 percent minocycline in 80 percent (v/v) MeOH and 20 percent (v/v) THF; or
1 percent CHA and 1 percent CHX and 1 percent triclosan (T) in 80 percent (v/v) MeOH and 20 percent (v/v) THF.

The various catheters were then tested for bacterial adherence. The luminal surfaces of all of the test catheters were continuously perfused with saline containing 10% TSB for 7 days and then locked with 1×10⁷ cfu/ml of S. aureus or E. aerogenes cultures for 24 hours. The catheters (body, extension lines and hubs) were then assessed for bacterial adherence by cutting the catheters with 1 cm segments, suspending each segment in 4 ml LTSB and sonicating them for 20 minutes. Thereafter, 0.5 ml of the LTSB was seeded on TSA plates or DE plates.

Results. The results are presented in Tables XXV, XXVI and XXVII.

TABLE XXV
Luminal Adherence of S. aureus (cfu/cm)

| | Catheters Perfused for 7 Days | | |
|---|---|---|---|
| Catheter Group (n) | Body | Extension Lines | Hub |
| Control (2) | 5.8 × 10³ | 3.2 × 10⁴ | 8.0 × 10⁴ |
| 1.2% CHA + 1.2% | 0 | 3 | 4.5 × 10² |
| CHX + 80% | | | |
| MeOH* + 20% | | | |
| THF* | | | |
| 1% CHA + 1% | 1.5 | 2.5 | 6.0 × 10² |
| CHX + 1% | | | |
| T + 80% | | | |
| MeOH* + 20% | | | |
| THF* (2) | | | |
| 2% CHX + 1% | 0 | 0 | 0 |
| M + 80% | | | |
| MeOH* + 20% | | | |
| THF* (2) | | | |

TABLE XXVI
Luminal Adherence of S. aureus (cfu/cm)

Catheters Perfused
for 11 Days

| Catheter Group (n) | Extension Lines | Hub |
|---|---|---|
| Control (3) | 5.7 × 10³ | 6.0 × 10⁴ |
| 1.2% CHA + 1.2% | 2 | 3.7 × 10³ |
| CHX + 80% | | |
| MeOH* + 20% | | |
| THF* (3) | | |
| 2% CHX + 1% | 0 | 5.4 × 10² |
| M + 80% | | |
| MeOH* + 20% | | |
| THF* (3) | | |

TABLE XXVII
Luminal Adherence of E. aerogenes cfu/cm

| | Catheters Perfused for 16 Days | | |
|---|---|---|---|
| Catheter Group (n) | Body | Extension Lines | Hub |
| Control (2) | 5.0 × 10⁵ | 5.0 × 10⁵ | 5.0 × 10⁵ |
| 1.2% CHA + 1.2% | 0 | 0 | 1.0 × 10⁴ |
| CHX + 80% | | | |
| MeOH* + 20% | | | |
| THF* (3) | | | |
| 1% CHA + 1% | 0 | 35 | 6.3 × 10³ |
| CHX + 1% | | | |
| T + 80% | | | |
| MeOH* + 20% | | | |
| THF* (3) | | | |
| 2% CHX + 1% | 0 | 0 | 4.4 × 10³ |
| M + 80% | | | |
| MeOH* + 20% | | | |
| THF* (3) | | | |

Discussion. Based on the data presented in Tables XXV, XXVI and XXVII, the catheters treated with minocycline plus chlorhexidine appear to have the lowest bacterial adherences, particularly at the hub. In Table XXVI, the adherence of S. aureus was found to be 1 log lower of colonies on catheters treated with minocycline plus chlorhexidine than on the other catheter groups.

### Example

### Long-Term Anti-adhesive Effects on Peripherally Inserted Central Catheters

Methods. Peripherally inserted central catheters (PICCs) were studied for long-term efficacy against S. aureus. The external surfaces of these polyurethane PICC catheters were treated with one of the following: 2 percent CHA and 1.5 percent CHX and 0.75 percent AgSD and 3% 93A polyurethane and 1 percent 60D polyurethane in 80 percent (v/v) MeOH and 20 percent (v/v) THF; or 2 percent CHA and 1 percent CHX and 1 percent M and 3 percent 93A polyurethane and 1 percent 60D polyurethane in 80 percent (v/v) MeOH and 20 percent (v/v) THF. The luminal surfaces of separate polyurethane PICC catheters were treated with one of the following solutions for 100 seconds: 1.2 percent CHA and 1.2 percent CHX in 80 percent (v/v) MeOH and 20 percent (v/v) THF; or 1 percent M and 2 percent CHX in 80 percent (v/v) MeOH and 20 percent (v/v) THF.

The external surfaces of the catheters were assayed according to the previously described agar tract in vitro model. The luminal surface of all of the test catheters were assayed as described in the prior example, except the lumen of the test catheters were exposed to the treatment solution by perfusion for 100 seconds.

The various catheters were tested for bacterial adherence as described in the agar tract in vitro model described in prior examples, except that after 12 days the catheters were transferred to fresh media and incubated with 1×10⁷ cfu/ml of S. aureus suspension. Bacterial adherence was evaluated as previously described 7 days after infection.

Results. The results are presented in Tables XXVIII and

TABLE XXVIII
Adherence of S. aureus (cfu/cm)

| Catheter Group | Catheters Perfused for 12 Days |
|---|---|
| None (Control) | 3.0 × 10³ |
| 2% CHA + 1.5% | 1.2 × 10² |
| CHX + 0.75% | |
| AgSD + 3% | |
| 93A + 1% | |
| 60D | |
| 2% CHA + 1% | 0 |
| CHX + 1% | |
| M + 3% | |
| 93A + 1% | |
| 60D | |

TABLE XXVIV
Luminal Adherence S. aureus (cfu/cm) in PICC Catheters

| | Catheter | | | |
|---|---|---|---|---|
| Catheter Group | No. | Body | Extension Line | Hub |
| 1.2% CHA + 1.2% | 1 | 0 | 2 | 1.3 × 10³ |
| CHX in | | | | |
| 80% MeOH + 20% | | | | |
| THF | | | | |
| 1.2% CHA + 1.2% | 2 | 0 | 1 | 9.6 × 10³ |
| CHX in | | | | |
| 80% MeOH + 20% | | | | |
| THF | | | | |
| 1% CHX + 1% | 1 | 0 | 0 | 1 |
| M in | | | | |
| 80% MeOH + 20% | | | | |
| THF | | | | |
| 1% CHX + 1% | 2 | 0 | 7 | 8 |
| M in | | | | |
| 80% MeOH + 20% | | | | |
| THF | | | | |
| Control | 1 | 1.0 × 10³ | 1.4 × 10³ | 7.2 × 10² |
| Control | 2 | 8.0 × 10² | 8.9 × 10² | 8.0 × 10⁴ |

Discussion: Based on the data presented in Tables XXVIII and XXVIV, the catheters treated with minocycline plus chlorhexidine diacetate plus chlorhexidine free base appear to have the lowest bacterial adherence to both external and luminal surfaces, particularly at the hub.

Various publications are cited herein, the contents of which are hereby incorporated in their entireties.

1. An anti-infective medical article prepared by exposing a polymer-containing medical article, for an effective period of time, to a treatment solution comprising between 1 and 8 percent (weight/volume) of minocycline and between 1 and 8 percent (weight/volume) of a chlorhexidine compound.

2. The anti-infective medical article of claim 1, where the treatment solution further comprises a bismuth salt at a concentration of between 0.5 and 2.0 percent (weight/volume).

3. The anti-infective medical article of claim 1, where the treatment solution further comprises between 0.2 and 1.0 percent (weight/volume) benzalkonium chloride.

4. The anti-infective medical article of claim 2, where the treatment solution further comprises between about 0.25 and 1.0 percent (weight/volume) benzalkonium chloride.

5. (canceled)

6. (canceled)

7. The anti-infective medical article of claim 2 where the bismuth salt is bismuth citrate.

8. The anti-infective medical article of claim 4, where the bismuth salt is bismuth citrate.

9. The anti-infective medical article of claim 2, where the bismuth salt is bismuth salicylate.

10. The anti-infective medical article of claim 4, where the bismuth salt is bismuth salicylate.

11. The anti-infective medical article of claim 1, where the chlorhexidine compound is selected from the group consisting of chlorhexidine gluconate, a mixture of chlorhexidine gluconate and chlorhexidine free base, a mixture of chlorhexidine gluconate and chlorhexidine diacetate, a mixture of chlorhexidine gluconate, chlorhexidine free base and chlorhexidine diacetate, and a mixture of chlorhexidine free base and chlorhexidine diacetate.

12. The anti-infective medical article of claim 2, where the chlorhexidine compound is selected from the group consisting of chlorhexidine gluconate, a mixture of chlorhexidine gluconate and chlorhexidine free base, a mixture of chlorhexidine gluconate and chlorhexidine diacetate, a mixture of chlorhexidine gluconate, chlorhexidine free base and chlorhexidine diacetate, and a mixture of chlorhexidine free base and chlorhexidine diacetate.

13. The anti-infective medical article of claim 3, where the chlorhexidine compound is selected from the group consisting of chlorhexidine gluconate, a mixture of chlorhexidine gluconate and chlorhexidine free base, a mixture of chlorhexidine gluconate and chlorhexidine diacetate, a mixture of chlorhexidine gluconate, chlorhexidine free base and chlorhexidine diacetate, and a mixture of chlorhexidine free base and chlorhexidine diacetate.

14. The anti-infective medical article of claim 4, where the chlorhexidine compound is selected from the group consisting of chlorhexidine gluconate, a mixture of chlorhexidine gluconate and chlorhexidine free base, a mixture of chlorhexidine gluconate and chlorhexidine diacetate, a mixture of chlorhexidine gluconate, chlorhexidine free base and chlorhexidine diacetate, and a mixture of chlorhexidine free base and chlorhexidine diacetate.

15. An anti-infective medical article prepared by exposing a polymer-containing medical article, for an effective period of time, to a treatment solution comprising between 1 and 8 percent (weight/volume)of minocycline, between 1 and 8 percent (weight/volume) of triclosan, and a bismuth salt at a concentration of between 0.5 and 2.0 percent (weight/volume).

16. The anti-infective medical article of claim 15, where the treatment solution further comprises between 0.25 and 1.0 percent (weight/volume) benzalkonium chloride.

17. (canceled)

18. (canceled)

19. The anti-infective medical article of claim 15, where the bismuth salt is bismuth citrate.

20. The anti-infective medical article of claim 16, where the bismuth salt is bismuth citrate.

21. The anti-infective medical article of claim 15, where the bismuth salt is bismuth salicylate.

22. The anti-infective medical article of claim 16, where the bismuth salt is bismuth salicylate.

23. An anti-infective medical article prepared by exposing a polymer-containing medical article, for an effective period of time, to a treatment solution comprising between 1 and 8 percent (weight/volume) of minocycline, between 0.25 and 1.0 percent (weight/volume) of benzalkonium chloride, and between 0.5 and 2.0 percent (weight/volume) of a bismuth salt.

24. The anti-infective medical article of claim 23, where the bismuth salt is selected from the group consisting of bismuth citrate, and bismuth salicylate.

25. An intravascular catheter comprising between 100 and 450 micrograms of minocycline per centimeter and between 130 and 520 micrograms of a chlorhexidine compound.

26. The catheter of claim 25 further comprising between 50 and 300 micrograms per centimeter of a bismuth salt.

27. The catheter of claim 26 where the bismuth salt is selected from the group consisting of bismuth citrate and bismuth salicylate.

28. The catheter of claim 26 further comprising between 25 and 100 micrograms per centimeter of benzalkonium chloride.

29. The catheter of claim 25 where the chlorhexidine compound is selected from the group consisting of chlorhexidine gluconate, a mixture of chlorhexidine gluconate and chlorhexidine free base, a mixture of chlorhexidine gluconate and chlorhexidine diacetate, a mixture of chlorhexidine gluconate, chlorhexidine free base and chlorhexidine diacetate, and a mixture of chlorhexidine free base and chlorhexidine diacetate.

30. The catheter of claim 25 further comprising between 50 and 200 micrograms per centimeter of a zinc salt.

31. The catheter of claim 25 further comprising between 25 and 300 micrograms per centimeter of a silver-containing compound.

32. (canceled)

33. An intravascular catheter comprising between 100 and 450 micrograms of minocycline per centimeter, between 130 and 750 micrograms of triclosan per centimeter, and between 50 and 300 micrograms of a bismuth salt per centimeter.

34. The catheter of claim 33 where the bismuth salt is selected from the group consisting of bismuth citrate and bismuth salicylate.

35. The catheter of claim 33 further comprising between 25 and 100 micrograms per centimeter of benzalkonium chloride.

36. The catheter of claim 33 further comprising between 50 and 200 micrograms per centimeter of a zinc salt.

37. The catheter of claim 33 further comprising between 25 and 300 micrograms per centimeter of a silver-containing compound.

38. (canceled)

39. An anti-infective medical article prepared by exposing a polymer-containing medical article for an effective period of time to a treatment solution comprising between 1 and 8 percent (weight/volume) of minocycline and between 0.5 and 2.0 percent (weight/volume) of a bismuth salt.
